(19) United States
(12) Patent Application Publication
(10) Pub. No.: US 2026/0218280 A1
(43) Pub. Date: Jul. 30, 2026

(54) COMPOUNDS AND METHODS FOR RNA SENSING

(71) Applicant: SAN DIEGO STATE UNIVERSITY RESEARCH FOUNDATION, San Diego, CA (US)

(72) Inventors: Byron W. PURSE, San Diego, CA (US); Marc Benjamin TURNER, Middle River, MD (US)

(73) Assignee: SAN DIEGO STATE UNIVERSITY (SDSU) FOUNDATION, DBA SAN DIEGO STATE UNIVERSITY RESEARCH FOUNDATION, San Diego, CA (US)

(21) Appl. No.: 19/147,190

(22) PCT Filed: Jan. 12, 2024
(86) PCT No.: PCT/US2024/011457

§ 371 Date: Jul. 10, 2025

Related U.S. Application Data
(60) Provisional application No. 63/479,838, filed on Jan. 13, 2023.

(51) Int. Cl. C12Q 1/6841; C07H 21/04

(52) U.S. Cl. CPC C12Q 1/6841; C07H 21/04

(57) ABSTRACT

The sequence-specific fluorescent detection of RNA using a tricyclic cytidine analogue such as ^{DEA}tC included as a surrogate for natural cytidine in DNA probe strands and that reports directly on Watson-Crick base pairing. The ^{DEA}tC-containing DNA oligonucleotide probes exhibit an average 8-fold increase in fluorescent intensity when hybridized to matched-RNA with ^{DEA}tC base paired with G, and no fluorescence turn-on when ^{DEA}tC is base paired with A. Time resolved fluorescence measurements in buffered H₂O vs D₂O show that ^{DEA}tC's fluorescence turn-on is primarily the result of protection against excited-state proton transfer in the matched hybrid duplex. Stern-Volmer quenching experiments and computational studies indicate that the combination of greater π stacking and narrower grooves in the A-form DNA-RNA heteroduplex provides additional shielding and favorable electronic interactions between bases, explaining why ^{DEA}tC's fluorescence turn-on response to an RNA target is about 3-fold greater than for DNA targets.

### RELATED APPLICATIONS

This application claims priority under 35 U.S.C. § 119 (e) to U.S. Provisional Patent Application No. 63/479,838, filed Jan. 13, 2023, which is incorporated herein by reference.

### BACKGROUND OF THE INVENTION

Fluorescent labeling methods for RNA have many applications in tracking, locating, and quantifying RNA in fixed and living cells. Metabolic labeling for RNA imaging can be performed using reactive nucleoside analogues such as 5-ethynyluridine and azido nucleosides, which can be click labeled following incorporation, or using intrinsically fluorescent nucleoside analogues for direct RNA imaging without staining. Artificially induced uptake, trafficking and function of exogenously produced RNAs can be monitored by including minimally perturbing fluorescent base analogues when these RNAs are prepared. But the most widely used applications of fluorescent imaging in RNA biology involve the detection of specific target sequences in cells or tissues.

Fluorescent hybridization probes recognize target sequences by base pairing. They can either be designed such that excess probe must be washed away prior to imaging, or they can provide a fluorescent response specific to their targets. Fluorescence in situ hybridization (FISH) typically involves the displacement of a quencher from a fluorescently labeled oligonucleotide probe, giving a turn-on response. Forced intercalation (FIT) probes are single-stranded nucleic acids or peptide nucleic acids with an intercalative fluorophore (e.g. thiazole orange) substituted for one nucleobase. Upon duplex or triplex formation with single- or double-stranded targets, respectively, the fluorophore is induced to intercalate, greatly increasing its fluorescence. Molecular beacons are oligonucleotide hairpins with a fluorophore appended to one terminus and a quencher appended to the other. They exhibit target-specific fluorescence turn-on upon hairpin opening, driven by the formation of a more stable hybrid duplex with the target. Fluorogenic aptamers are frequently used in place of fluorophores for RNA imaging in cells. The aptamer's ability to exchange fluorogenic ligands overcomes the problem of photobleaching. CRISPR-based methods using catalytically inactive dCas13 can also be used to image endogenous RNAs using a guide RNA to provide target specificity.

One of the most important limitations of these probing schemes is that their sequence-specificity is determined primarily by the relative affinity for a matched vs. mismatched RNA target. The greater stability of GC as compared with AT base pairs creates bias, and sequences with a single mismatch may have only slightly depressed binding affinity as compared with a perfectly matched complement. Accordingly, many of these probes are prone to false positives, especially in GC-rich sequences or in samples with a high abundance of off-target RNAs. An attractive alternative is to develop fluorescent probes that reported directly on RNA sequence by altering their fluorescence in response to base pairing.

Fluorescent nucleobase analogues (FBAs) are widely used in studies on the structure, dynamics, and biomolecular interactions of nucleic acids, and they have great potential to address the challenge of sequence-specific nucleic acid detection. Because they can be present at the Watson-Crick interface, their fluorescence can report directly on the identity of the base pairing partner. Since the discovery of 2-aminopurine's (2AP) fluorescence in 1969, more than 100 fluorescent nucleoside analogues have been reported. Intrinsically fluorescent nucleobase analogues have been used to report on local structure, conformation, and dynamics of nucleic acids, sequence (mis) matches, enzyme-mediated nucleobase modifications, and changes in local polarity or pH. Some nucleobase analogues such as 2AP experience nearly complete emission quenching when base-stacked in double-stranded nucleic acids, however others may remain emissive when base-stacked, including the largely environmentally insensitive tricyclic cytidine (tC) nucleobase.

The problem is sequence-specific fluorescence probes are lacking and advancements in this field are needed.

### SUMMARY

By expanding on the parent tC molecular scaffold with a series of chemical modifications to the original phenothiazine scaffold, we have developed novel derivatives with environmentally sensitive fluorescent properties. One of these compounds, the tricyclic 2′-deoxycytidine analogue ^{DEA}tC, is nearly non-emissive as a free nucleoside in aqueous solution (Φₑₘ=0.006) but experiences up to a 20-fold fluorescence turn-on effect (up to Φₑₘ=0.12) when base-stacked in double-stranded DNA (dsDNA) and base-paired with guanine (FIG. 1), with some dependence on the identity of neighboring bases. This fluorescence turn-on response to base pairing and stacking is rare; a few other base analogues that exhibit significant fluorescent increases in response to duplex formation have been reported and their turn-on results for local rigidification (J. Am. Chem. Soc. 2017, 139, 1372-1375).

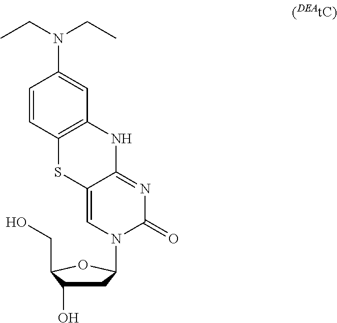

Here, we investigate the fluorescence turn-on response of ^{DEA}tC to DNA-RNA heteroduplex formation, showing that its fluorescence increase is more than two-folder greater than in DNA-DNA homoduplex formation and with less sensitivity to the identity its base-stacked neighbors. NMR structure determine of a 10-mer dsDNA duplex including ^{DEA}tC shows that it extends into the major groove but does not significantly perturb duplex structure. Time-resolved fluorescence measurements and Stern-Volmer quenching measurements indicate that the enhanced performance of ^{DEA}tC in DNA-RNA is attributable primarily to altered electronic interactions with stacked bases as compared with DNA-DNA, a consequence of the A-form conformation of the heteroduplex.

Accordingly, this disclosure provides a nucleic acid comprising formula I:

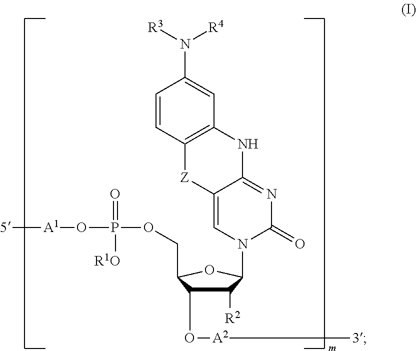

wherein
A¹ is a nucleotide comprising guanine (G), adenine (A), cytosine (C), thymine (T), or a combination thereof;
A² is a nucleotide comprising cytosine (C), adenine (A), guanine (G), thymine (T) or a combination thereof;
R¹ is H or —(C₁-C₆)alkyl;
R² is H or OH;
R³ and R⁴ are each independently —(C₁-C₆)alkyl or H; or
   R³ and R⁴ taken together with the nitrogen atom to which they are attached form a 3- to 8-membered heterocycloalkyl, wherein the heterocycloalkyl is unsubstituted or substituted;
Z is S or O;
5′ and 3′ are each independently oligo- or polynucleotides; and m is 1 to about 100.

Also, this disclosure provides a method for differentiating nucleic acid sequences, comprising:
a) hybridizing the nucleic acid of described above to a complementary DNA or RNA sequence to form a homoduplex- or heteroduplex-nucleic acid;
b) detecting a fluorescent signal; and
c) quantifying the fluorescent signal, wherein the formed homoduplex- or heteroduplex-nucleic acid increases the fluorescent signal in comparison to a single stranded form of formula IA;
wherein the DNA or RNA sequence can thereby be differentiated from a non-complementary sequence of DNA or RNA.

The invention provides novel compounds of formula I, formula IA or IB, intermediates for the synthesis of compounds of formula I, formula IA or IB, as well as methods of preparing compounds of formula I, formula IA or IB. The invention also provides compounds of formula I, formula IA or IB that are useful as intermediates for the synthesis of other useful compounds.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Structure and fluorescent properties of the ^{DEA}tC fluorescent nucleoside analogue. Absorption and emission spectra are shown for a 10-mer DNA strand containing an internal 5′ GXC 3′ sequence, where X is ^{DEA}tC. Fluorescence turn-on is observed upon hybridization with a DNA or RNA complement. Fluorescence spectra are scaled to Φₑₘ at λₘₐₓ.

FIG. 2. Fluorescence quantum yields of ^{DEA}tC-containing 10-mers. Sequence names are given in Table 1. ssDNA=^{DEA}tC-containing 10mer as a single-stranded oligonucleotide. dsDNA refers to those ^{DEA}tC-containing 10-mers hybridized to a complementary 10-mer DNA sequence. DNA-RNA refers to those ^{DEA}tC-containing 10-mers hybridized to a complementary 10-mer RNA sequence. Error bars indicate the standard deviation of the mean. For CXA dsDNA, CXC dsDNA, GXG dsDNA, GXG DNA-RNA, and CXT DNA-RNA standard deviations were <2.5×10⁻³. Measurements were made in 1×PBS buffer at pH 7.4.

FIG. 3. Fidelity of ^{DEA}tC's fluorescence turn-on. Fluorescent emission of the AXA probe strand hybridized to complementary DNA or RNA strands with ^{DEA}tC:G, ^{DEA}tC:I, and ^{DEA}tC:A pairings is shown. Emission intensity (Φₑₘ) is normalized to that of ^{DEA}tC:G in dsDNA. The normalized fluorescent intensity of the AXA probe strand is shown as a baseline for the absence of turn-on (bottom curve).

FIG. 4. Deoxyribose H1′ to aromatic portion of the NOESY spectra of GCC′ (A) at 27° C. and GXC′ (B) at 15° C., both at a mixing time of 300 ms. The sequential connectivity for H1′ to H6/H8 for residues 1-10 is indicated by a grey line, while that for residues 11-20 is indicated by a black line.

FIG. 5. Superposition of family of 20 structures describing the GCC′ duplex (A) obtained by rMD; superposition of the family of 20 structures describing the GXC′ duplex (B) obtained by rMD, and a comparison of the average structures for GCC′ (C) and GXC′ (D).

FIG. 6. Time-resolved emission decays of various probes with ^{DEA}tC:G basepairs (uppermost lines) along with mispairings of the AXA probe with ^{DEA}tC:A (lowermost line) and ^{DEA}tC:I (middle line).

FIG. 7A-C. Stern-Volmer analysis of GXC duplexes. A) Fluorescence was measured as a function of [Q] and integration of emission spectra enabled graphical determination of Stern-Volmer quenching coefficients. B) Attenuated emission was recorded with varying [NaCl], and C) [NaI].

FIG. 8A-H. Time-resolved emission decays of ^{DEA}tC DNA-RNA duplexes. Emission intensity within 250-650 nm is measured by TCSPC using an excitation of 371 nm. Measurements were made in 1×PBS buffer at pH 7.4.

FIG. 9A-C. Time-resolved emission decays of ^{DEA}tC duplexes of AXA paired across guanosine, mispaired across adenosine, and mispaired across inosine. The duplexes with guanosine and adenosine were DNA-RNA and the duplex with inosine was DNA-DNA. Emission intensity within 250-650 nm is measured by TCSPC using an excitation of 371 nm. Measurements were made in 1×PBS buffer at pH 7.4.

FIG. 10A-D. Circular dichroism spectra and melting curves of ^{DEA}tC DNA-RNA sequences. Spectra were recorded at 25° C. with 2.5 μM sample duplex in 1×PBS buffer at pH 7.4.

FIG. 11A-B. Stern-Volmer analysis of sequences TXA and CXA. Experiments were performed using sodium chloride or sodium iodide.

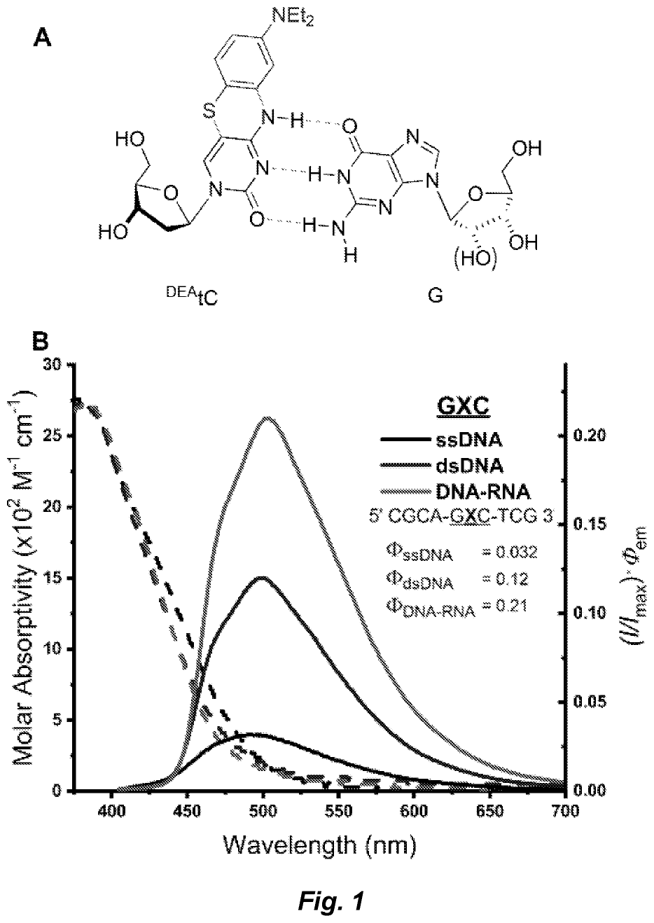

### DETAILED DESCRIPTION

Sequence-specific fluorescent probes for RNA are widely used in microscopy applications such as FISH and a growing number of newer approaches to live-cell RNA imaging. The sequence specificity of most of these approaches relies on differential hybridization of the probe to the correct target. Competing sequences with only one or two base mismatches are prone to causing off-target recognition. Here, we report the sequence-specific fluorescent detection of model RNA targets using a tricyclic cytidine analogue ^{DEA}tC that is included as a surrogate for natural cytidine in DNA probe strands and that reports directly on Watson-Crick base pairing.

The ^{DEA}tC-containing DNA oligonucleotide probes exhibit an average 8-fold increase in fluorescent intensity when hybridized to matched RNA with ^{DEA}tC base paired with G, and little fluorescence turn-on when ^{DEA}tC is base paired with A. Duplex structure determination by NMR, time-resolved fluorescence studies and Stern-Volmer quenching experiments suggest that the combination of greater π stacking and narrower grooves in the A-form DNA-RNA heteroduplex provides additional shielding and favorable electronic interactions between bases, explaining why ^{DEA}tC's fluorescence turn-on response to RNA targets is typically three-fold greater than for DNA targets.

Additional information and data supporting the invention can be found in the following publication by the inventors: Bioconjugate Chem. 2023, 34, 1061-1071 and its Supporting Information, which are incorporated herein by reference in its entirety.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability, necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

The recitation of a), b), c), . . . or i), ii), iii), or the like in a list of components or steps do not confer any particular order unless explicitly stated.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

This disclosure provides methods of making the compounds and compositions of the invention. The compounds and compositions can be prepared by any of the applicable techniques described herein, optionally in combination with standard techniques of organic synthesis. Many techniques such as etherification and esterification are well known in the art. However, many of these techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6; as well as standard organic reference texts such as March's Advanced Organic Chemistry: Reactions, Mechanisms, and Structure, 5th Ed., by M. B. Smith and J. March (John Wiley & Sons, New York, 2001); Comprehensive Organic Synthesis. Selectivity, Strategy & Efficiency in Modern Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983); for heterocyclic synthesis see Hermanson, Greg T., Bioconjugate Techniques, Third Edition, Academic Press, 2013.

The formulas and compounds described herein can be modified using protecting groups. Suitable amino and carboxy protecting groups are known to those skilled in the art (see for example, Protecting Groups in Organic Synthesis, Second Edition, Greene, T. W., and Wutz, P. G. M., John Wiley & Sons, New York, and references cited therein; Philip J. Kocienski; Protecting Groups (Georg Thieme Verlag Stuttgart, New York, 1994), and references cited therein); and Comprehensive Organic Transformations, Larock, R. C., Second Edition, John Wiley & Sons, New York (1999), and referenced cited therein.

The term “halo” or “halide” refers to fluoro, chloro, bromo, or iodo. Similarly, the term “halogen” refers to fluorine, chlorine, bromine, and iodine.

The term “alkyl” refers to a branched or unbranched hydrocarbon having, for example, from 1-20 carbon atoms, and often 1-12, 1-10, 1-8, 1-6, or 1-4 carbon atoms; or for example, a range between 1-20 carbon atoms, such as 2-6, 3-6, 2-8, or 3-8 carbon atoms. As used herein, the term “alkyl” also encompasses a “cycloalkyl”.

The term “heteroatom” refers to any atom in the periodic table that is not carbon or hydrogen. Typically, a heteroatom is O, S, N, P. The heteroatom may also be a halogen, metal or metalloid.

The term “heterocycloalkyl” or “heterocyclyl” refers to a saturated or partially saturated monocyclic, bicyclic, or polycyclic ring containing at least one heteroatom selected from nitrogen, sulfur, oxygen, preferably from 1 to 3 heteroatoms in at least one ring. Each ring is preferably from 3- to 10-membered, more preferably 4 to 7 membered.

As used herein, the term “substituted” or “substituent” is intended to indicate that one or more (for example, in various embodiments, 1-10; in other embodiments, 1-6; in some embodiments 1, 2, 3, 4, or 5; in certain embodiments, 1, 2, or 3; and in other embodiments, 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound. Suitable indicated groups include, e.g., alkyl, alkenyl, alkynyl, alkoxy, haloalkyl, hydroxyalkyl, aryl, heteroaryl, heterocyclyl, cycloalkyl, alkanoyl, alkoxycarbonyl, amino, alkylamino, dialkylamino, carboxyalkyl, alkylthio, alkylsulfinyl, and alkylsulfonyl. Substituents of the indicated groups can be those recited in a specific list of substituents described herein, or as one of skill in the art would recognize, can be one or more substituents selected from alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, and cyano.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof, such as racemic mixtures, which form part of the present invention. Many organic compounds exist in optically active forms, i.e., they have the ability to rotate the plane of plane-polarized light. In describing an optically active compound, the prefixes D and L, or R and S. are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate (defined below), which may occur where there has been no stereoselection or stereospecificity in a chemical reaction or process.

The term “hybridizing” as referred to herein means the formation of a double stranded nucleic acid, such as DNA-DNA, RNA-RNA, or DNA-RNA.

### Statements of the Technology

1. This disclosure provides a nucleic acid comprising formula I:

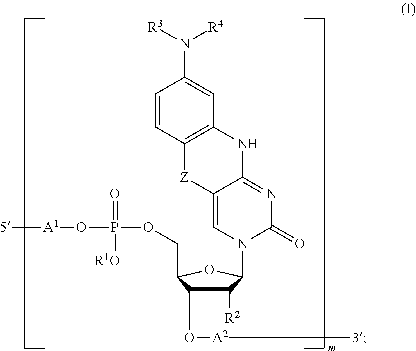

wherein
   A¹ is a nucleotide comprising guanine (G), adenine (A), cytosine (C), thymine (T), or a combination thereof;
   A² is a nucleotide comprising cytosine (C), adenine (A), guanine (G), thymine (T) or a combination thereof;
   R¹ is H or —(C₁-C₆)alkyl;
   R² is H or OH;
   R³ and R⁴ are each independently —(C₁-C₆)alkyl or H; or
      R³ and R⁴ taken together with the nitrogen atom to which they are attached form a 3- to 8-membered heterocycloalkyl, wherein the heterocycloalkyl is unsubstituted or substituted;
   Z is S or O;
   5′ and 3′ are each independently an oligo- or polynucleotide wherein the number of nucleotide units in the oligo- or polynucleotide is about 10 to about 25, or greater than 25; and
   m is 1, 2, 3, 4, 5, 6, 7, 8, 9, 10, 10-100, or greater than 100, for example, up to about 1,000, about 10,000, or about 100,000.
2. The nucleic acid of statement 1, wherein R³ and R⁴ are both methyl, ethyl, or propyl.
3. The nucleic acid of statement 1 or 2, wherein R³ and R⁴ taken together with the nitrogen atom to which they are attached form:

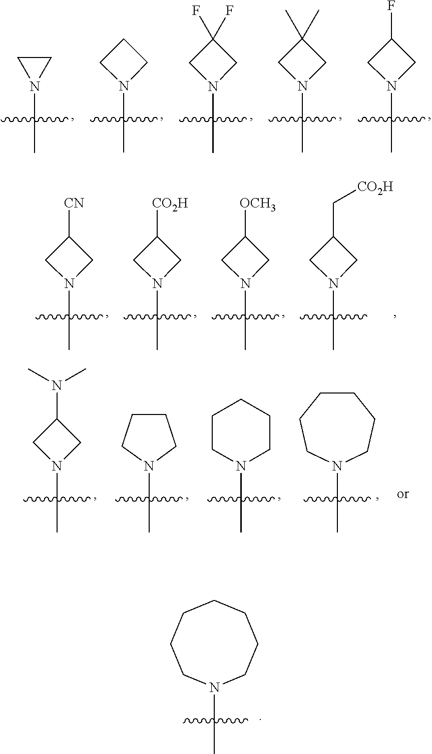

4. The nucleic acid of statement 1, wherein formula I is represented by formula IA:

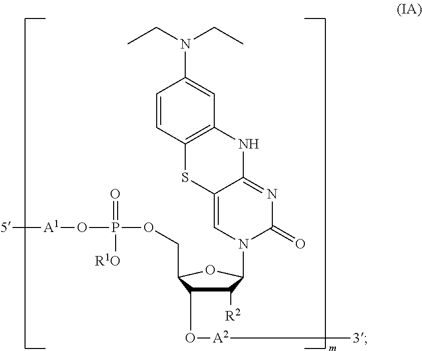

wherein
   A¹ is a nucleotide comprising adenine (A), cytosine (C), guanine (G), thymine (T), or a combination thereof;
   A² is a nucleotide comprising adenine (A), cytosine (C), guanine (G), thymine (T) or a combination thereof;
   R¹ is H or —(C₁-C₆)alkyl;
   R² is H or OH;
   5′ and 3′ are each independently an oligo- or polynucleotide wherein the number of nucleotide units in the oligo- or polynucleotide is about 10 to about 25, or greater than 25; and
   m is 1, 2, 3, 4, 5, 6, 7, 8, 9, 10, 10-100, or greater than 100, for example, up to about 1,000, about 10,000, or about 100,000.
5. The nucleic acid of any one of statements 1-4, wherein R¹ and/or R² are/is H.
6. The nucleic acid of any one of statements 1-5, wherein formula I or IA is represented by formula IB:

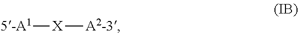

wherein X is the moiety between A¹ and A² of formula I or IA:

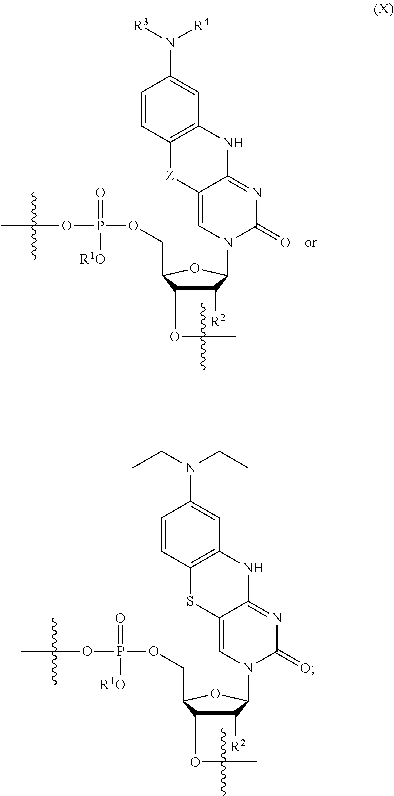

and
   the nucleic acid comprises: GXC, GXA, TXA, CXA, CXC, AXA, GXG, or CXT.
7. The nucleic acid of statement 6, wherein the nucleic acid comprises:

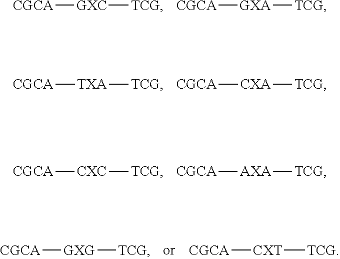

8. The nucleic acid of any one of statements 1-5, wherein formula I or IA is represented by formula IB:

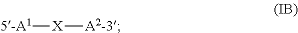

wherein, X (shown above) is the moiety between A¹ and A² of formula I or IA; and
   the nucleic acid comprises: TXG, GXT, CXG, AXG, AXT, AXC, TXC, or TXT.
9. The nucleic acid of any one of statements 1-5, wherein:
A¹ is G and A² is C; A¹ is G and A² is A; A¹ is T and A² is A; A¹ is C and A² is A;
A¹ is C and A² is C; A¹ is A and A² is A; A¹ is G and A² is G; A¹ is C and A² is T;
A¹ is T and A² is G; A¹ is G and A² is T; A¹ is C and A² is G; A¹ is A and A² is G;
A¹ is A and A² is T; A¹ is A and A² is C; A¹ is T and A² is C; or A¹ is T and A² is T.
7. The nucleic acid of any one of statements 1-5, wherein A¹ comprises G and A² comprises A, C, G, T, or a combination thereof.
8. The nucleic acid of any one of statements 1-5, wherein A¹ comprises C and A² comprises A, C, G, T, or a combination thereof.
9. The nucleic acid of s any one of statements 1-5, wherein A¹ comprises T and A² comprises A, C, G, T, or a combination thereof.
10. The nucleic acid of any one of statements 1-5, wherein, A¹ comprises A and A² comprises A, C, G, T, or a combination thereof.
11. The nucleic acid of any one of statements 1-5, wherein A² comprises G and A¹ comprises A, C, G, T, or a combination thereof.
12. The nucleic acid of any one of statements 1-5, wherein A² comprises C and A¹ comprises A, C, G, T, or a combination thereof.
13. The nucleic acid of any one of statements 1-5, wherein A² comprises T and A¹ comprises A, C, G, T, or a combination thereof.
14. The nucleic acid of any one of statements 1-5, wherein A² comprises A and A¹ comprises A, C, G, T, or a combination thereof.
15. The nucleic acid of any one of statements 1-14, wherein m is about 5 to about 25, about 25 to about 50, about 50 to about 75, about 75 to about 100, or about 100 to about 150.
17. The nucleic acid of any one of statements 1-14, wherein nucleic acid comprises or consists of about 5 to about 25 nucleotides, about 25 to about 50 nucleotides, about 50 to about 100 nucleotides, or about 100 to about 500 nucleotides.
18. A composition comprising the nucleic acid of anyone of statements 1-17 and DNA or RNA, wherein the nucleic acid is hybridized to the DNA or RNA at complementary sites or sequences that form or are capable of forming a homoduplex or heteroduplex, respectively.
19. A method for differentiating nucleic acid sequences, comprising:
   a) hybridizing the nucleic acid of any one of statements 1-17, to a complementary DNA or RNA sequence to form a homoduplex- or heteroduplex-nucleic acid;
   b) detecting a fluorescent signal; and
   c) quantifying the fluorescent signal, wherein the formed homoduplex- or heteroduplex-nucleic acid increases the fluorescent signal in comparison to a single stranded form of formula I, formula IA or IB;
   wherein the DNA or RNA sequence can thereby be differentiated from a non-complementary sequence of DNA or RNA.
20. The method of statement 19, wherein the method further comprises:
   d) differentiating the complementary DNA or RNA sequence from a non-complementary sequence of DNA or RNA.
21. The method of statement 19 or 20, wherein formula I or IA is represented by formula IB:

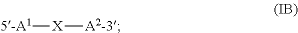

wherein, X (shown above) is the moiety between A¹ and A² of formula I or IA; and
   the nucleic acid comprises: GXC, GXA, TXA, CXA, CXC, AXA, GXG, or CXT.
22. The method of statement 19 or 20, wherein:
A¹ is G and A² is C; A¹ is G and A² is A; A¹ is T and A² is A; A¹ is C and A² is A;
A¹ is C and A² is C; A¹ is A and A² is A; A¹ is G and A² is G; A¹ is C and A² is T;
A¹ is T and A² is G; A¹ is G and A² is T; A¹ is C and A² is G; A¹ is A and A² is G;
A¹ is A and A² is T; A¹ is A and A² is C; A¹ is T and A² is C; or A¹ is T and A² is T.
23. The method of any one of statements 19-22, wherein the tricyclic cytidine moiety of formula IA is based-paired with guanosine or a guanine base of the complementary DNA or RNA sequence.
24. The method of any one of statements 19-23, wherein a heteroduplex-nucleic acid is formed with a complementary RNA sequence.
25. The method of any one of statements 19-24, wherein the quantified fluorescence has an increase in emission quantum yield (Φₑₘ) of about 2-fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, about 50-fold, or higher.
26. A method for detecting acidity in a sample or determining pH in a sample, comprising:
   a1) contacting the sample and the nucleoside ^{DEA}tC:

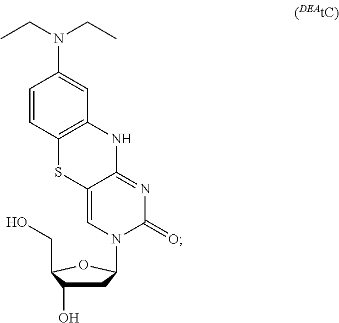

or
a2) contacting the sample and a nucleic acid comprising formula IA or formula IB of any one of statements 1-12;
b) quantifying a fluorescent signal when present in the contacted sample; and
c) determining the acidity and/or pH of the contacted sample.
27. The method of statement 26 wherein the sample is a feature, structure, site, or position that is in a cell.

In some embodiments, ^{DEA}tC exhibits very low fluorescence as a nucleoside in a PBS solution. In some embodiments, guanine has the lowest reduction potential, −2.74 V, of the four canonical DNA bases (G<A<C<T).

In some embodiments, the N,N-diethylamine moiety of formula IA or IB is replaced by other N,N-dialkylamines wherein the alkyl moieties of the N,N-dialkylamine are each independently (C₁-C₅)alkyl, unbranched or branched. Further embodiments include the compounds, methods, and techniques described in U.S. Pat. No. 11,447,519 (Purse et al.) and US Publication No. 2022/0088226 (Purse et al.), which are incorporated herein by reference. For example, the compound of formula IA or IB of this disclosure can be replaced by formula I of U.S. Pat. No. 11,447,519 in any embodiment described in this disclosure wherein the formula I is conjugated to an oligo- or polynucleotide. Furthermore, the compound of formula IA or IB of this disclosure can be replaced by any of the formulas of US Publication No. 2022/0088226 in any embodiment described in this disclosure wherein Z of formula I is an oligo- or polynucleotide.

As would be readily recognized by one of skill in the art, the number of nucleotide units in the oligo- or polynucleotide moieties 5′ and 3′ can vary and will depend upon the type of oligo- or polynucleotide used for the preparation of the nuclei acid comprising formula I. In various embodiments, the number of nucleotide units in the oligo- or polynucleotide moieties 5′ and 3′ can be the number of nucleotides found in a known nuclei acid and in some embodiments, at least 10, at least 25, at least 50, at least 100, at least 250, at least 500, at least 1,000, or at least 10,000, and/or less than 100,000, less than 10,000, less than 5,000, less than 1,000, less than 500, less than 250, or less than 100, and/or a range between any two of the aforementioned integers, for example, 25 to 1,000 or 100 to 5,000.

### Results.

Synthesis of ^{DEA}tC and design and preparation of oligonucleotide probes. ^{DEA}tC and its corresponding dimethoxytrityl-protected phosphoramidite were synthesized as reported previously (Chem.-Eur. J. 2019, 25 (5), 1249-1259). To measure ^{DEA}tC's fluorescent properties in DNA-RNA heteroduplexes and compare with similar measurements in DNA-DNA homoduplexes, we selected a set of eight 10-mer sequences with varied 3′- and 5′-neighboring nucleobases that we and others have used previously to study FBAs (Table 1). The ^{DEA}tC amidite was incorporated into these sequences using standard solid-phase synthesis conditions, except the coupling time for the ^{DEA}tC amidite was increased 10-fold. The identity and purity of the sequences was confirmed by mass spectrometry and HPLC. We named the sequences according to the identity of the 3′- and 5′-neighbors. For example, GXC refers to the sequence 3′-CGCAGXCTCG-5′, where X is ^{DEA}tC.

TABLE 1
Steady-State fluorescence measurements from ^{DEA}tC in DNA-RNA
duplexesª.

| | | | Stokes | | | |
|---|---|---|---|---|---|---|
| DNA Sequence | λ_{max,} _{abs} | λ_{max,} ₑₘ | Shift | | | Brightness |
| (5′→3′) | (nm) | (nm) | (nm) | Φₑₘ (±) | ΔTₘ (±)^{b} | (M⁻¹ cm⁻¹)^{c} |
| CGCA-GXC-TCG | 404 | 504 | 100 | 0.22 (7.4 × 10−3) | −7.05 (0.77) | 594 |
| CGCA-GXA-TCG | 394 | 501 | 107 | 0.17 (1.4 × 10−2) | −2.83 (1.8) | 459 |
| CGCA-TXA-TCG | 409 | 503 | 94 | 0.14 (2.4 × 10−2) | +0.61 (0.60) | 378 |
| CGCA-CXA-TCG | 410 | 497 | 87 | 0.13 (5.2 × 10−2) | −3.39 (0.85) | 351 |
| CGCA-CXC-TCG | 411 | 494 | 83 | 0.12 (3.9 × 10−2) | −6.92 (0.64) | 324 |
| CGCA-AXA-TCG | 392 | 502 | 110 | 0.11 (1.6 × 10−2) | −1.16 (1.0) | 297 |
| CGCA-GXG-TCG | 402 | 503 | 101 | 0.10 (2.7 × 10−4) | −5.43 (0.54) | 270 |
| CGCA-CXT-TCG | 410 | 499 | 89 | 0.048 (6.9 × 10−4) | +1.49 (1.1) | 130 |

^{a}The position of ^{DEA}tC is denoted by X. Sequence names are in bold. Complementary RNA sequences are matched.
^{b}Duplex stability is calculated by subtracting Tₘ (°C) of duplexes with natural cytidine from Tₘ (° C.) of duplexes substituting ^{DEA}tC at position X.
^{c}Brightness is calculated using ϵ = 2,700 M⁻¹ cm⁻1 at 395 nm. Measurements were made in 1 × PBS buffer at pH 7.4.

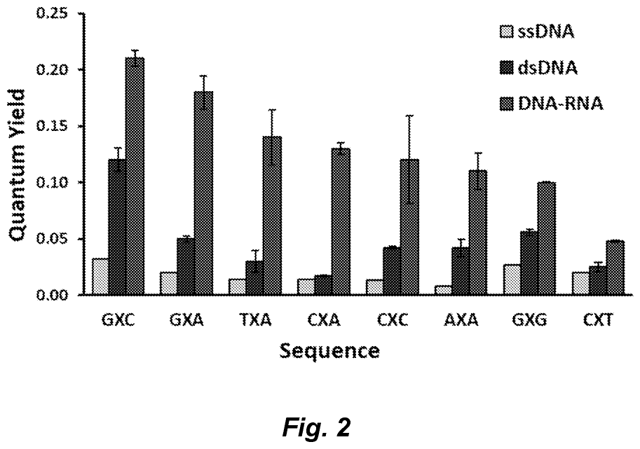

Fluorescence of ^{DEA}tC in single-stranded and duplex oligonucleotides. ^{DEA}tC has low fluorescence as a free nucleoside, with an emission quantum yield Φₑₘ=0.006 (λ_{max,abs}=395 nm and λ_{max,em}=493 nm) in 1×PBS buffer at pH 7.4. This fluorescence is approximately two- to three-fold brighter when ^{DEA}tC is present in single-stranded DNA oligonucleotides (FIG. 2 and Table 5). A much greater increase in fluorescence, up to Φₑₘ=0.12, occurs upon hybridization to a matched DNA complement, but not when ^{DEA}tC is mispaired with adenine or present opposite an abasic site (Table 6). The magnitude of this Φₑₘ increase is influenced by the 3′- and 5′-neighbors of ^{DEA}tC. Some sequences, such as AXA, exhibit up to a 5-fold fluorescence increase upon hybridization to form a matched dsDNA duplex. In these sequences, the single-stranded ^{DEA}tC-containing oligonucleotide probe has especially low fluorescence. The brightest sequences have guanosine as the 5′-neighbor of ^{DEA}tC, but these exhibit only a 2- to 3-fold fluorescence turn-on upon matched hybridization.

Here, we measured the steady-state fluorescence of ^{DEA}tC in DNA-RNA heteroduplexes using the same 10-mer DNA oligonucleotide sequences by hybridizing them to RNA complements. Consistently greater ^{DEA}tC fluorescence intensity is observed in the DNA-RNA duplexes, with GXC remaining the most emissive sequence and CXT the least (Table 1 and FIG. 2). GXC exhibited the greatest Φₑₘ=0.22, a 6.9-fold increase with respect to the GXC 10-mer ssDNA and a 37-fold increase with respect to the ^{DEA}tC nucleoside. Most other sequences had Dem values ranging from 0.10-0.17 (Table 1). CXT exhibited the lowest Φₑₘ=0.048, but was still 2.4-fold brighter than the ssDNA probe strand prior to hybridization. The AXA sequence exhibits the largest fluorescence enhancement, a 14-fold increase with respect to the ^{DEA}tC-containing ssDNA oligo, upon hybridization with complementary RNA. CXA shows the largest difference in turn-on enhancement when hybridized to RNA as compared with DNA. Brightness [B=ε·Φ] is approximated using ε₃₉₅=2,700 M⁻¹ cm⁻¹ for ^{DEA}tC, although the molar absorptivity of DNA bases usually decreases 25-40% in oligonucleotides compared to free nucleoside monomers. The exact decrease in ^{DEA}tC's absorptivity upon inclusion in an oligonucleotide has not been measured. The Stokes shifts among the heteroduplexes are largely consistent, ranging from 83-110 nm. The excitation energy as determined by λ_{max,abs} ranges from 3.02-3.16 eV, which is very similar to the range from 2.94-3.12 eV observed for ^{DEA}tC in dsDNA. These excitation energies are slightly lower than the range for parent tC, 3.11-3.19 eV in dsDNA. The excitation energy of ^{DEA}tC as a free nucleoside is 3.14 eV. Similar excitation energies for ^{DEA}tC when base paired and stacked in a dsDNA or DNA-RNA indicates that the energy gap between the ground (S₀) and excited states (S₁) remains relatively constant.

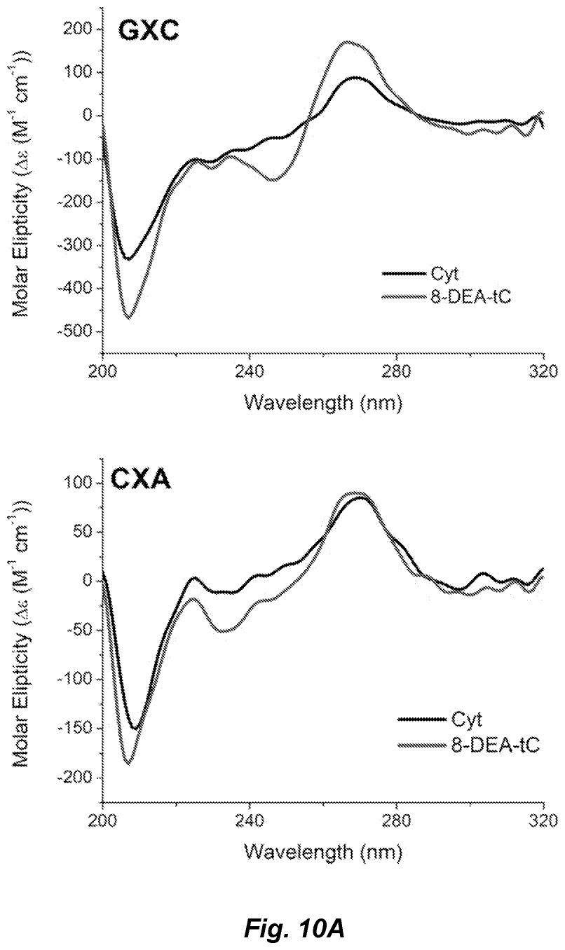

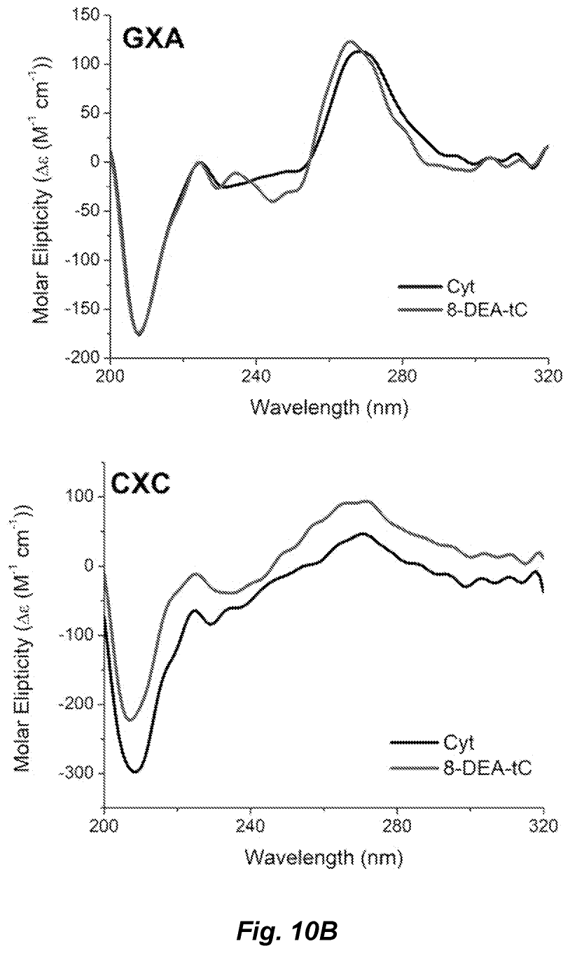

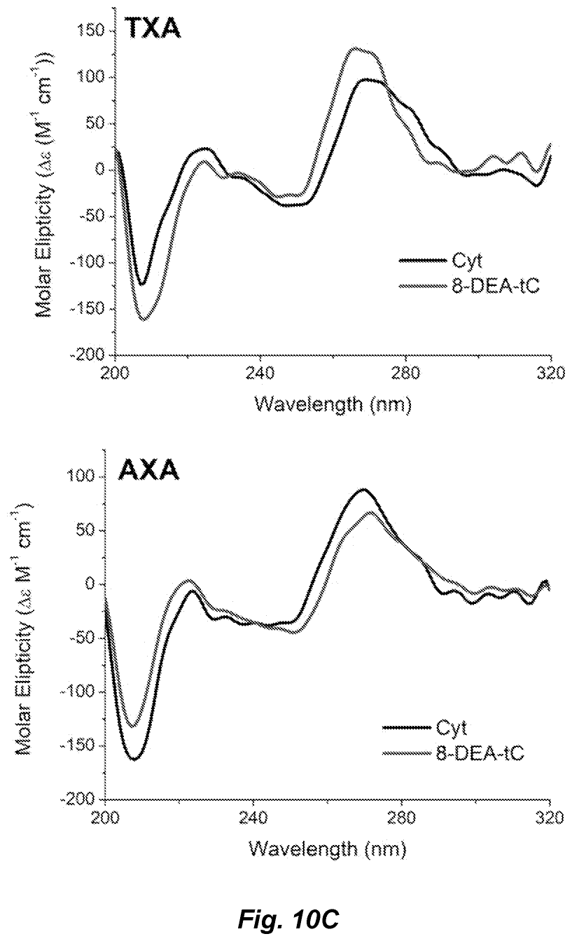

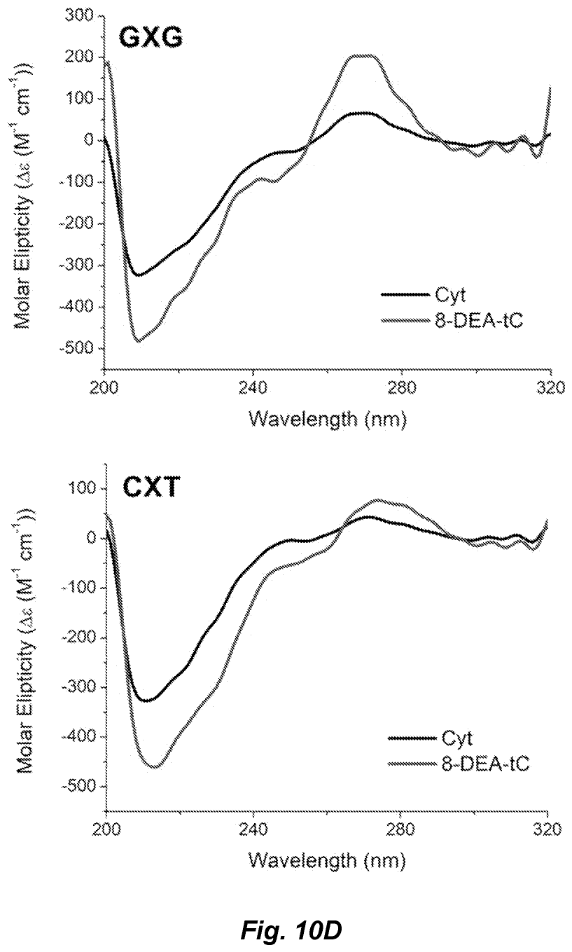

The stability of the DNA-RNA heteroduplexes and some aspects conformational perturbation caused by ^{DEA}tC can be measured by ΔTₘ, the difference in melting temperature between the ^{DEA}tC-containing and native duplexes (Table 1). Depending on the identity of the neighboring bases, ΔTₘ varies between −7.0° C. (GXC) and +1.5° C. (CXT). In six out of the eight tested sequences, ^{DEA}tC is typically moderately destabilizing. The corresponding values for ^{DEA}tC-containing DNA-DNA homoduplexes are a range from −14.5° C. (GXC) to +6.7° C. (CXC). ^{DEA}tC is, on average, more destabilizing in DNA-DNA duplexes. This result could be considered surprising, given that DNA-RNA heteroduplexes take the A-form conformation, in contrast with B-form for DNA-DNA. The π system overlap in stacked neighboring bases is greater in the A-form. Circular dichroism measurements show that the A-form is retained in DNA-RNA and the B-form retained in DNA-DNA when ^{DEA}tC substitutes for one cytidine in the sequences studied here (FIG. 10).

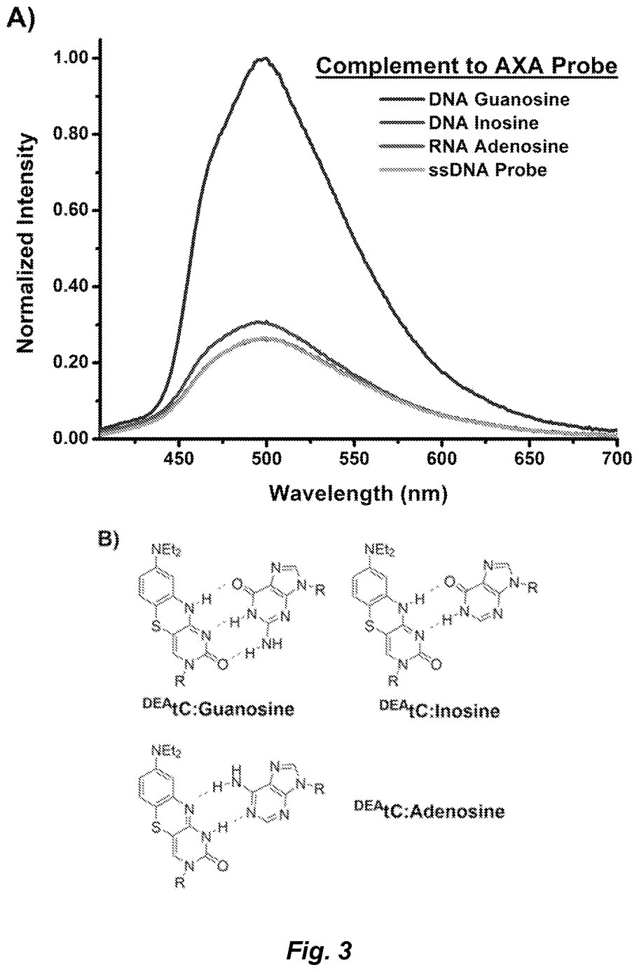

Our past studies on ^{DEA}tC in duplex DNA oligonucleotides show that its fluorescence turn-on is dependent on matched base pairing with guanine. Opposite adenosine or an abasic site, ^{DEA}tC does not exhibit fluorescence turn-on. Consistent with that finding, ^{DEA}tC's fluorescence turn-on response to RNA is dependent on canonical Watson-Crick base-pairing with guanine. When the quantum yield was measured for a DNA-RNA ^{DEA}tC:A mismatch using the AXA sequence, the fluorescence was comparable to AXA ssDNA Φₑₘ, revealing no significant turn-on response (FIG. 3A). We performed a similar fidelity experiment with a DNA complement that included the non-canonical nucleoside inosine (FIG. 3B), which bears a hypoxanthine nucleobase. Inosine has a Watson-Crick hydrogen-bonding face partially resembling guanine and consequently base pairs with cytosine. I:C base-pairs differ from classic G:C base pairs in that hypoxanthine engages in two hydrogen bonds with cytosine, lacking an H-bond with the O2 acceptor on cytosine (FIG. 3B). Although inosine presents similar features to guanosine, we did not observe a significant fluorescence turn-on for a dsDNA ^{DEA}tC:I duplex. Engaging the three Watson-Crick H-bonding sites on ^{DEA}tC with a contraposing guanine is required for induced fluorescence turn-on.

NMR-based duplex conformational studies. We performed NMR structure determination on a ^{DEA}tC-containing DNA-DNA duplex and the corresponding native duplex to further study changes in structure and dynamics induced by this analogue. For these experiments, we selected a modified GXC sequence, chosen both because it is the brightest sequence studied and it has the most perturbed melting temperature. To facilitate NMR structure determination, we slightly modified the sequence to 5′-CGTA-GXC-TCC-3′, which we will call GXC′ (X=^{DEA}tC; c.f. the GXC sequence in Table 1). The altered sequence has 5′- and 3′-termini designed to minimize fraying, without changing ^{DEA}tC's closest neighboring bases. To verify that this sequence change does not significantly alter the fluorescence, we measured absorption and emission spectra of GXC′ and determined its Φₑₘ as a single-stranded oligo (Φₑₘ=0.021), in a matched DNA-DNA duplex (^{DEA}tC base paired with G; Φₑₘ=0.13), and in a DNA-DNA duplex with ^{DEA}tC base paired with A (Φₑₘ=0.041). These values are nearly the same as those observed for GXC, and they show that “mispairing” ^{DEA}tC with A does not induce a large fluorescence turn-on in this alternative set of neighboring bases. Accordingly, ^{DEA}tC's local chemical environment in this modified duplex is effectively the same as in the parent GXC sequence.

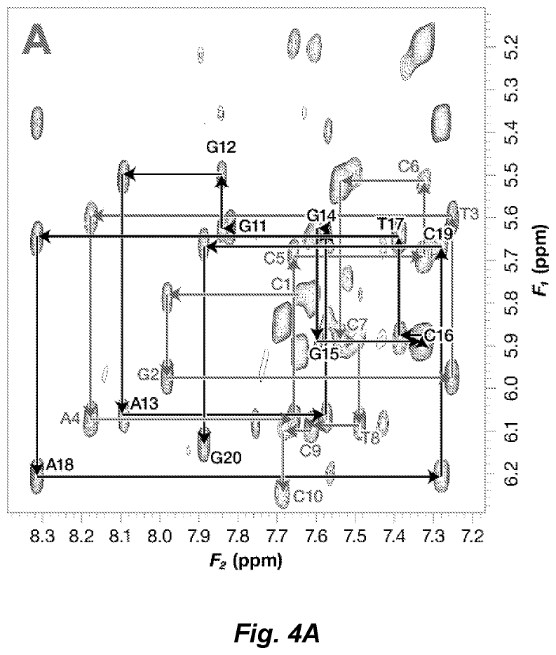

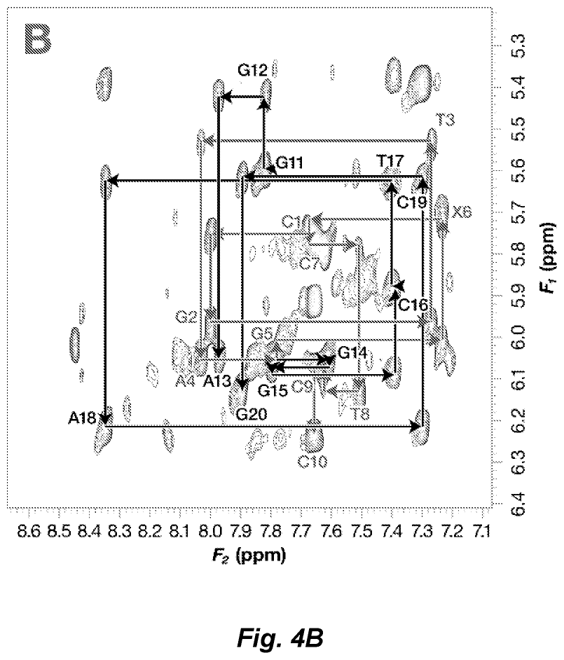

Signal assignments of all exchangeable and non-exchangeable nucleic acid protons of the GCC′ (the native duplex corresponding to GXC′) and GXC′ duplexes (with the exception of C5′H and C5″H) were made based on standard procedures. Imino N—H protons in the duplexes were visible at 27° C. (GCC′) and 15° C. (GXC′). FIG. 4 shows the sequential assignment of the aromatic base protons (H6/H8) and the C1′H of the DNA sequence in GCC′ (27° C.) and GXC′ (15° C.) in the NOESY spectrum collected in D₂O. Sequential connectivity was also observed for the aromatic protons with most of the C2′H and C2″H of the deoxyribose rings.

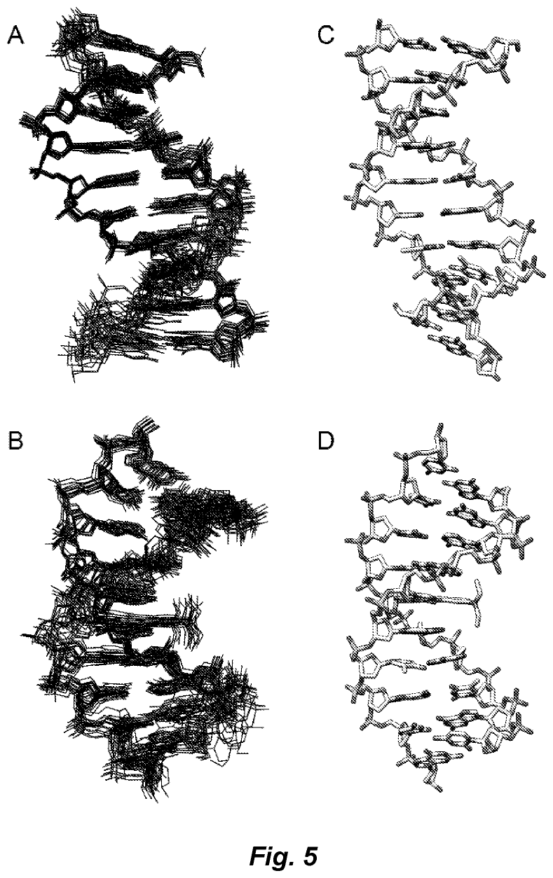

The families of structures used to represent each duplex were generated using statistical analysis modeled after Smith, et al. (J. Cell Sci. 2003, 116 (Pt 14), 2833-2838). The structures resulting from rMD for each duplex were randomly ordered and the mean all-atom pairwise rmsd was calculated for the first two structures, then the first three structures, etc. This process was repeated 500 times with each round using a different ordering of the structures. This analysis predicts the minimum number of structures necessary to fully represent the conformational space consistent with the experimental data. It was determined that 20 structures (GCC′) and 20 structures (GXC′) were sufficient to describe the duplexes. The structures in each family were chosen to minimize the molecular mechanics (AMBER) energy and the constraint violation energy. The superposition of the family of structures describing both duplexes is shown in FIG. 5 along with the average structures for the duplexes and the binding sites.

The energy and rmsd characteristics of the ensembles of structures for GCC′ and GXC′ are summarized in Table 2. The data indicate structural convergence for the GCC′ duplex with a rmsd of 1.02 Å (rms difference from the mean structure of 0.70 Å) and for the GXC′ duplex with a rmsd of 1.27 Å (rms difference from the mean structure of 0.88 Å), given that the starting structures for both duplexes represented a range of B-DNA conformations with initial rmsd values of 3.97 Å (GCC′) and 3.98 Å (GXC′). The final collection of structures has a total restraint violation summing to 7.6±0.4 kcal for GCC′ and 6.0±0.7 kcal for GXC′, amounting to 0.2% (GCC′) and 0.1% (GXC′) of the total energy of each system.

TABLE 2
Summary of energies, rmsd values and violations
for ensembles of structures.

| Molecular Mechanics energies (kcal): | GCC′ | GXC′ |
|---|---|---|
| E_{Amber} | −4493.3 ± 1.0 | −4476.9 ± 1.6 |
| Eᵥᵢₒₗ | 7.6 ± 0.4 | 6.0 ± 0.7 |

Average Pairwise rmds (Å):

| | | |
|---|---|---|
| DNA | 1.02 | 1.27 |

Distance violations (Å):

| | | |
|---|---|---|
| 0.05 < d ≤ 0.10 | 5 | 9 |
| 0.10 < d ≤ 0.20 | 0 | 2 |
| 0.20 ≤ d | 0 | 0 |

Helical analyses (using Curves5+ indicate that both GCC′ and GXC′ duplexes exhibit overall B-form DNA geometry. In the region of the ^{DEA}tC moiety there is no major disruption in the GXC′ duplex relative to the unmodified GCC′. There are minor differences in a few helical parameters such as roll, shift, stagger, tip, and γ-displacement likely due to dynamic motion as the duplex accommodates the diethylamino group in the major groove. Relative to the GCC′ duplex, the GXC′ duplex is more highly dynamic, as evidenced by the loss of signal (due to broadening) in key regions of the NOESY spectra including NOEs between protons on the DNA and the CH₃ of the diethylamino groups on ^{DEA}tC. These particular NOEs broaden but are visible at 10° C. (presumably from aggregation of the duplex—not unusual), sharp at 15° C., weakening at 20°, and not visible at 25° and 35° C. The observed increase in duplex dynamics when ^{DEA}tC substitutes for C in this sequence explains the lower melting temperature of GXC. We note that the diethylamino group of ^{DEA}tC is electron-donating and guanine is the most electron-rich canonical nucleobase. We propose that the forced π stacking of these electron-rich arenes upon duplex formation is destabilizing and drives the increased dynamics.

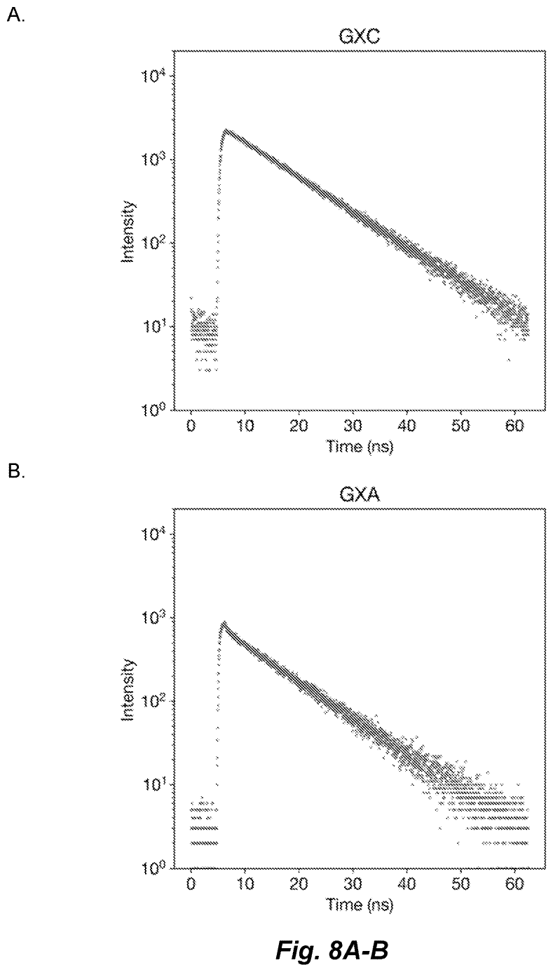

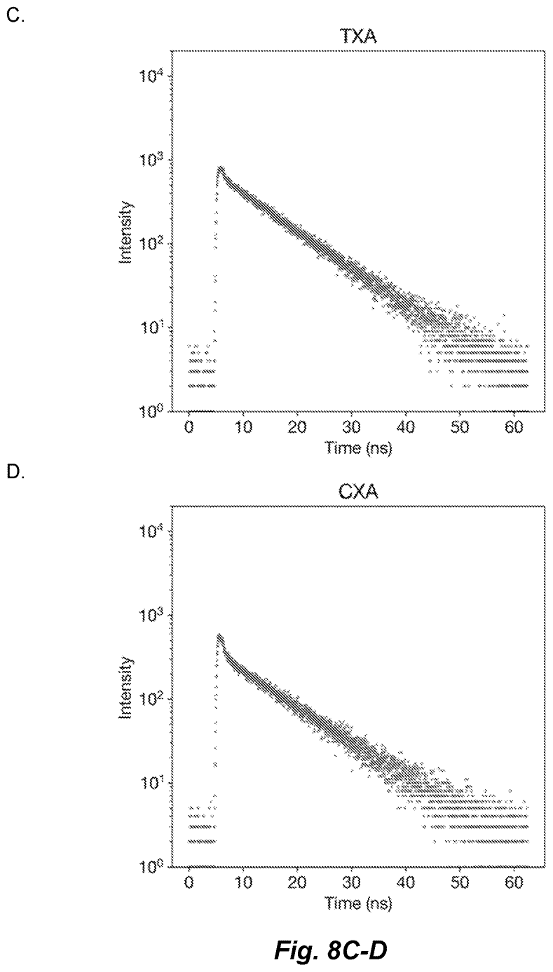

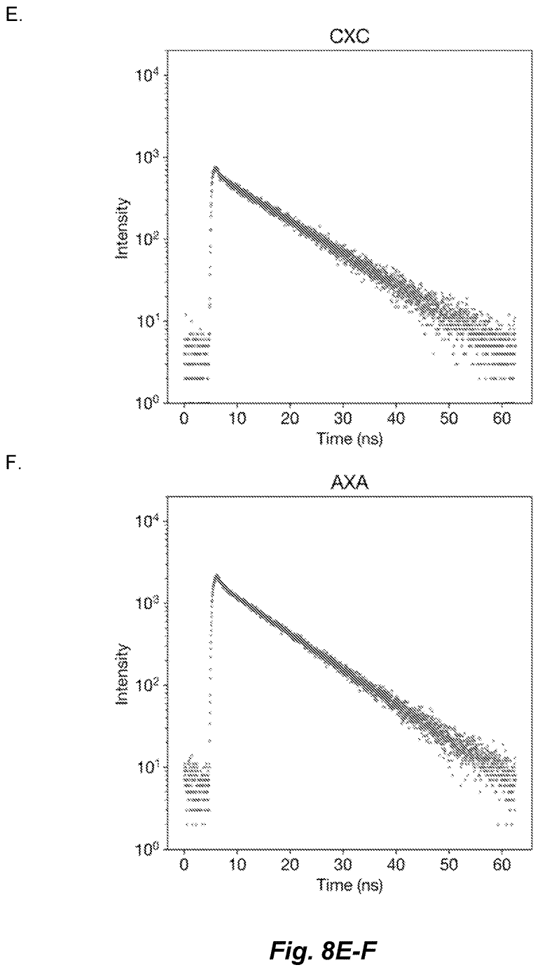

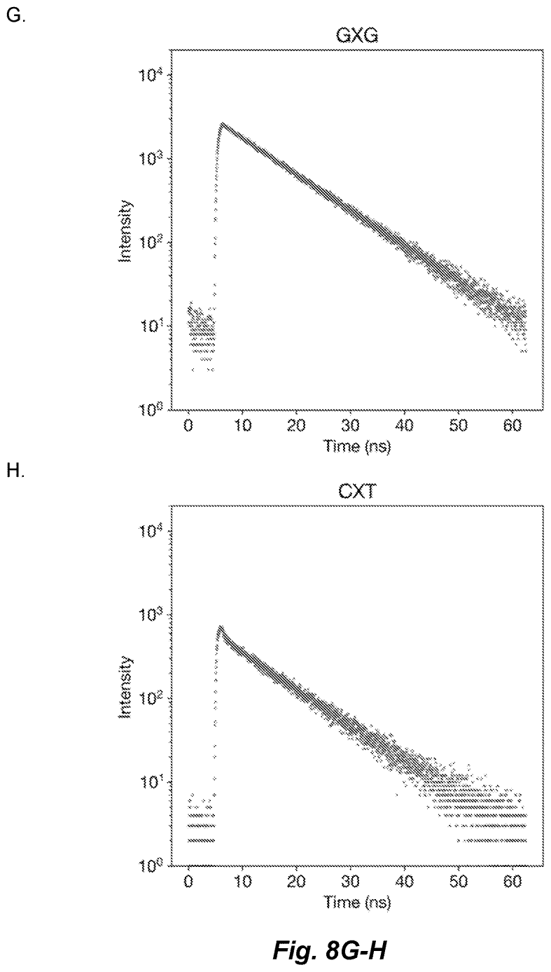

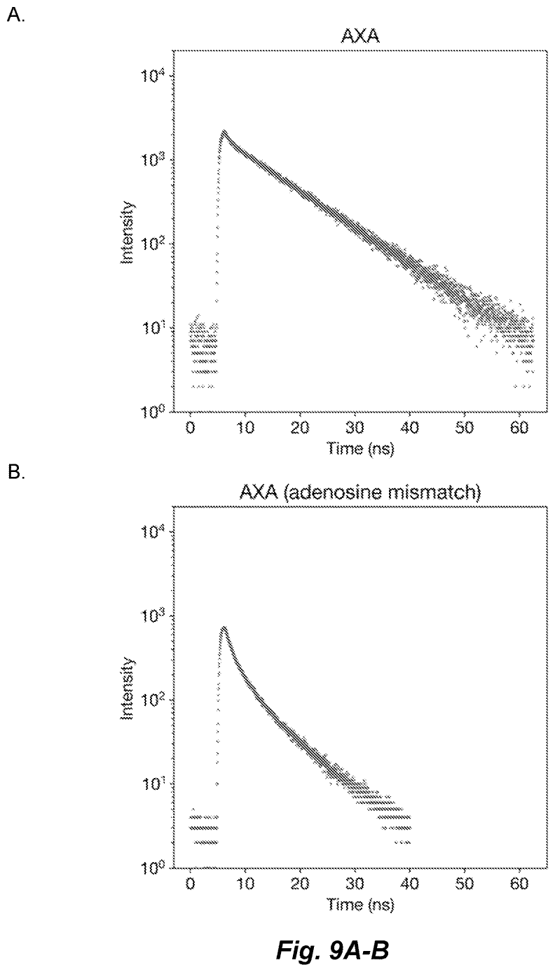

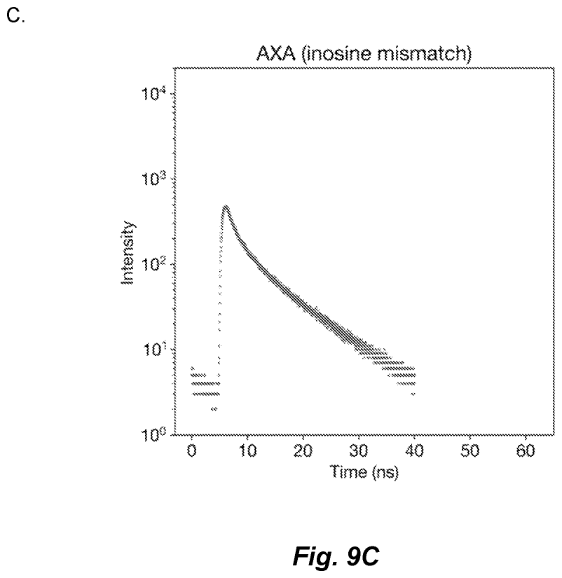

Time-resolved fluorescence measurements. To gain further insight into the origins of ^{DEA}tC's fluorescence turn-on response to matched RNA, we performed time-resolved fluorescence spectroscopy using time-correlated single photon counting (TCSPC) to measure the excited-state lifetimes t (FIG. 8, FIG. 9 and Table 3). These measurements were performed using a Delta Pro™ Fluorescence Lifetime System (Horiba Scientific) with a LED Delta Diode 371 nm excitation source, with samples dissolved in in 1×PBS buffer at pH 7.4. Data fitting to determine fluorescence lifetimes was performed using the maximum entropy method as implemented in the MemExp software (ver. 6.0). We calculated amplitude average fluorescence lifetimes <τ> and the radiative and nonradiative rate constants for relaxation, kᵣ and kₙᵣ, respectively.

TABLE 3
Time-resolved fluorescence measurements of ^{DEA}tC nucleoside in DNA-RNA duplexes.

| Sequence | Φₑₘ | τ₁ (ns) | α₁ | τ₂ (ns) | α₂ | <τ> (ns)^{d} | k_{f} (×10⁷ s⁻¹)^{e} | kₙᵣ (×10⁷ s⁻¹)^{f} |
|---|---|---|---|---|---|---|---|---|
| Nucleoside | 0.006 | 7.3 | 0.62 | 3.0 | 0.38 | 5.69 | 0.001 | 0.18 |
| GXC | 0.22 | 10.5 | 1.00 | — | — | 10.54 | 0.021 | 0.07 |
| GXA | 0.17 | 10.3 | 0.79 | 2.7 | 0.21 | 8.70 | 0.020 | 0.10 |
| TXA | 0.14 | 10.0 | 0.71 | 2.2 | 0.29 | 7.74 | 0.018 | 0.11 |
| CXA | 0.13 | 10.6 | 0.55 | 1.1 | 0.45 | 6.30 | 0.021 | 0.14 |
| CXC | 0.12 | 11.2 | 0.77 | 1.6 | 0.23 | 9.05 | 0.013 | 0.10 |
| AXA | 0.11 | 10.2 | 0.79 | 2.3 | 0.21 | 8.52 | 0.013 | 0.10 |
| GXG | 0.10 | 10.2 | 1.00 | — | — | 10.20 | 0.010 | 0.09 |
| CXT | 0.048 | 10.5 | 0.70 | 2.3 | 0.30 | 8.06 | 0.006 | 0.12 |
| AXA^{b} | n.d. | 8.5 | 0.19 | 2.2 | 0.81 | 3.37 | | |
| AXA^{c} | n.d. | 9.7 | 0.26 | 2.2 | 0.74 | 4.20 | | |

^{a}Time-resolved measurements were performed using TCSPC and λₑₓ = 371 nm. Sequences names are defined in Table 1. A single-component model were used for GXC and GXG because there was no clear evidence for a significantly shorter-lifetime component.
^{b}Hybridized to a complementary RNA strand with ^{DEA}tC base paired with adenosine.
^{c}Hybridized to a complementary DNA strand with ^{DEA}tC base paired with inosine.
^{d}Amplitude average fluorescence lifetime <τ> = Σαᵢτᵢ.
^{e}Radiative decay rate is calculated as Φₑₘ divided by <τ>.
^{f}Non-radiative decay rates were calculated using the equation kₙᵣ = (k_{f}/Φₑₘ) − k_{f}. n.d. = not determined.

In most observed contexts, the ^{DEA}tC nucleoside's excited state exhibits biexponential decay. The major component of this decay function has a lifetime τ=7.3 ns for the free nucleoside, and this lifetime increases to τ≈10 ns in DNA-RNA duplexes. A shorter lifetime of τ≈2 ns is observed in most duplexes as a minor component. The amplitude average fluorescence lifetime <τ> ranges from 6.3-10.5 ns for the duplexes and is not clearly correlated with any one fluorescence property such as Φₑₘ. Traditionally, a biexponential decay is interpreted to result from the fluorophore being present in two distinct environments. While it is not certain what those environments might be in this context, at least for these matched base pairs, one possibility is that an imino tautomer of ^{DEA}tC, which would be expected to be a minor component, could form a wobble base pair with G. Another possibility is that sequences exhibiting τ₂ have a minor conformer giving rise to this component. While these results do not clearly explain the origin of ^{DEA}tC's fluorescence turn-on effect, we do note that the lifetime of the major component increases from 7.3 to approximately 10 ns in the duplex, indicative of an environment that slows excited state relaxation (i.e. limits quenching).

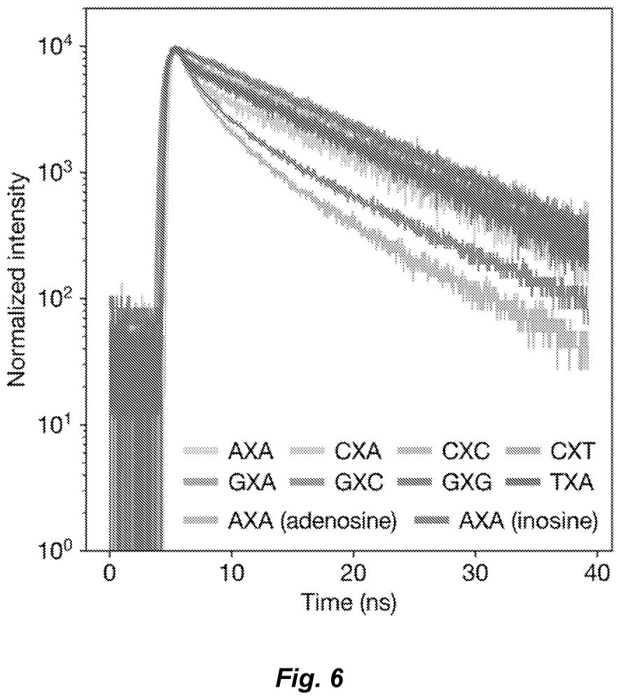

To further shed light on ^{DEA}tC's fluorescence turn-on, we performed additional TCSPC measurements with ^{DEA}tC base paired with inosine and adenosine, respectively. Here, we observed a large increase in the contribution of a short lifetime τ₂≈2 ns in a biexponential decay (FIG. 6). For these “mispairings”, the short lifetime has an amplitude of 0.74 and 0.81, respectively. In contrast, the amplitude of the short lifetime τ₂≈2 ns was typically close to 0.3 in the “matched” sequences (i.e. those with ^{DEA}tC:G base pairs); here, the long lifetime dominates. These results show that both the ^{DEA}tC:I and ^{DEA}tC:A base pairs are associated with major populations of ^{DEA}tC in environments conducive to rapid quenching.

Stern-Volmer studies on sensitivity to external quenchers. The general trends of ^{DEA}tC's brightness as depending on neighboring bases are similar in dsDNA and DNA-RNA duplexes, but the brightness is considerably greater in the latter. DNA-RNA hybrids typically adopt A-form conformation, similarly to dsRNA, which are structurally distinct from B-form. CD spectroscopy revealed that all of the ^{DEA}tC-containing sequences adopted the A-form when paired to their complementary 10-mer RNA (FIG. 10). These comparisons were performed by examining the CD spectral differences between sequences containing ^{DEA}tC to those containing natural cytosine and confirming the presence of a sharp transition at 210 nm. One possible explanation for ^{DEA}tC's greater brightness in DNA-RNA is reduced accessibility to external quenchers in the deeper but narrower major groove of the A-form conformation. A-form has a greater tilt of the base-pair plane with respect to the central helical axis, approximately 11 base pairs per turn instead of 10 in B-form, and a greater extent of base stacking. The increased overlap of the bases and narrower major groove can be expected to endow greater shielding to the nucleobases from external quenchers in DNA-RNA. We tested this hypothesis by performing Stern-Volmer quenching analysis using chloride and iodide, which are commonly used collisional quenchers.

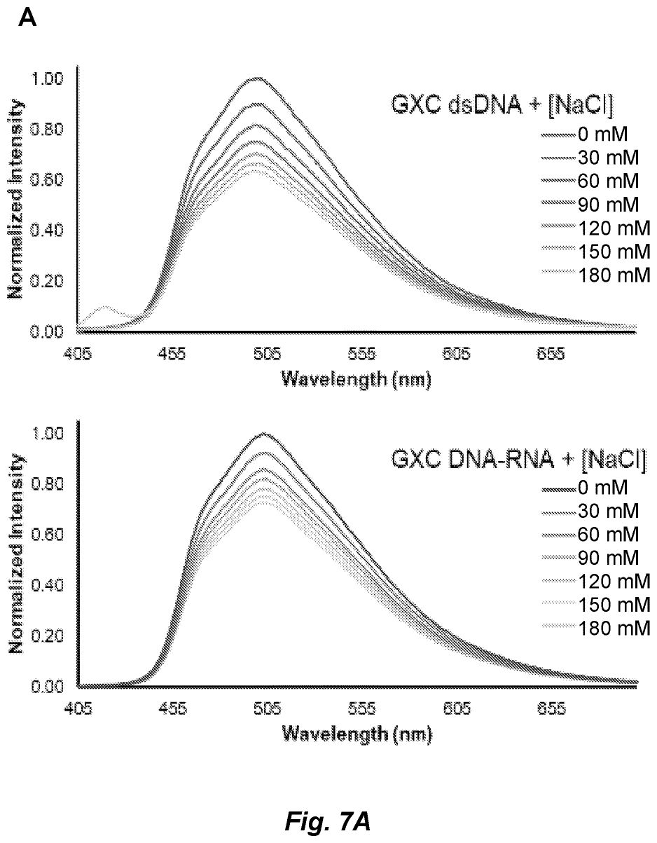

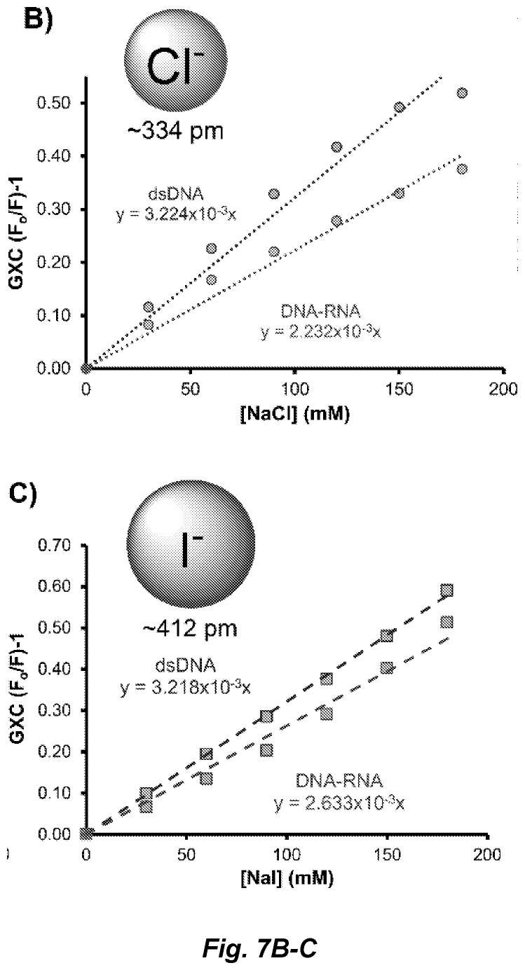

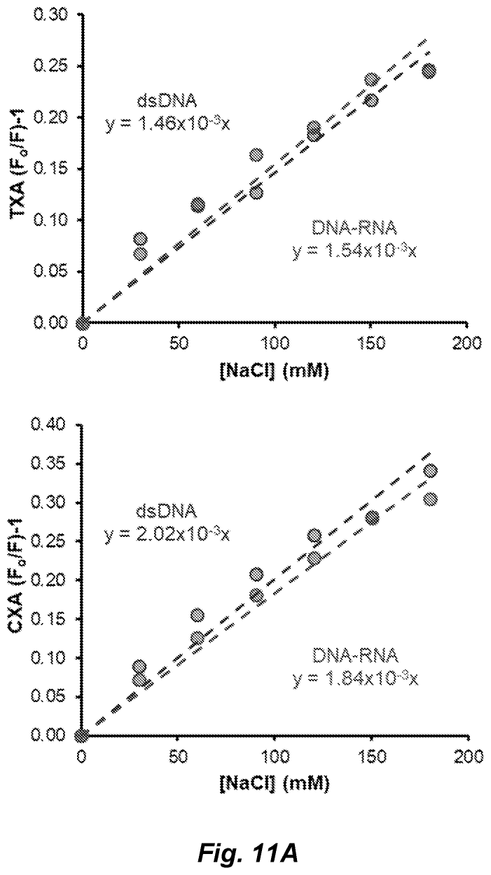

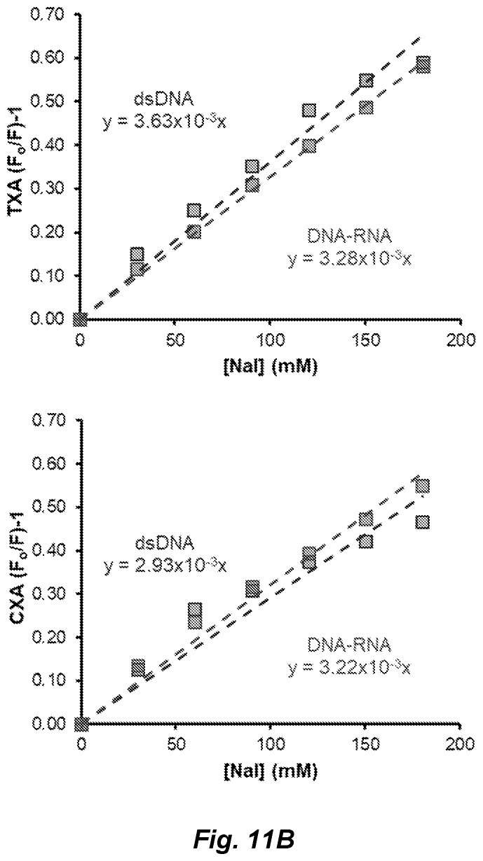

Stern-Volmer quenching experiments were performed with three sequences and two halide ions in sodium phosphate buffer (FIG. 7). The two halides, chloride and iodide, differ in their ionic radius as well as their quenching potency, which has been correlated to their ionization energies. Chloride has ionic diameter of about 334 pm while iodide has an ionic diameter of approximately 412 pm and is a stronger quencher. Experiments were performed using the DNA-RNA oligonucleotides GXC, TXA, and CXA (FIG. 4, FIG. 11, and Table 4). GXC was the brightest sequence observed while TXA and CXA showed the most pronounced fluorescence increase in A-form DNA-RNA duplexes relative to fluorescence in B-form dsDNA. Using the recorded amplitude average fluorescence lifetimes and Stern-Volmer quenching coefficients, we calculated the bimolecular quenching rate constants kg for the TXA and CXA sequences, comparing DNA-RNA with DNA-DNA. The results are that rate constants for quenching are greater for iodide, as expected, and that the rate constants for quenching are slower in the DNA-RNA duplexes. The difference is modest for the TXA sequence, only 5% slower quenching in DNA-RNA with chloride and 19% slower with iodine. The difference is much more in the CXA duplex, 53% slower with chloride and 43% slower iodide. While significant, we note that ^{DEA}tC's fluorescence turn-on upon matched duplex formation is 5-fold greater in DNA-RNA than in DNA-DNA in the TXA sequence, and 9-fold greater in CXA. Accordingly, less sensitivity to quenching by solutes in DNA-RNA contributes to ^{DEA}tC's greater fluorescence turn-on, but it is not the most important effect.

TABLE 4
Quenching efficiency measured by Stern-Volmer analysis^{a}.

| | | NaCl | | NaI | |
|---|---|---|---|---|---|
| Sequence | Duplex | K_{SV} (×10⁻³ M⁻¹) | k_{q} (×10⁸ M⁻¹ s⁻¹)^{b} | K_{SV} (×10⁻³ M⁻¹) | k_{q} (×10⁸ M⁻¹ s⁻¹)^{b} |
| GXC | dsDNA | 3.22 ± 0.13 | n.d. | 3.22 ± 0.23 | n.d |
| | DNA-RNA | 2.23 ± 0.072 | 2.12 | 2.63 ± 0.92 | 2.49 |
| TXA | dsDNA | 1.46 ± 0.072 | 1.11 | 3.63 ± 0.14 | 5.24 |
| | DNA-RNA | 1.54 ± 0.076 | 1.99 | 3.28 ± 0.38 | 4.24 |
| CXA | dsDNA | 2.02 ± 0.090 | 6.18 | 2.93 ± 0.18 | 8.96 |
| | DNA-RNA | 1.84 ± 0.057 | 2.92 | 3.22 ± 0.10 | 5.11 |

^{a}The Stern-Volmer quenching constant is graphically measured.
^{b}The bimolecular quenching constant (kg) is calculated from the relation K_{SV} = k_{q}τ.

### Discussion

The fluorescence of most FBAs is quenched in the base stack by photoinduced electron transfer with neighboring bases, but in the last twenty years, a number of FBAs were introduced that retain robust fluorescence emission upon incorporation into duplex nucleic acids, with little sensitivity to base pairing and stacking. More recently, some of the first FBAs have been reported to provide substantial fluorescence increases in response to specific base pairing and stacking interactions. These include ^{DEA}tC, for which we first reported a fluorescence turn-on responses to matched DNA, and ^{ts}T, a brightly fluorescent thymidine analogue with 5-fold fluorescence turn-on response that is specific for matched base pairing the adenosine in DNA-DNA duplexes. It is known that FBAs must have HOMO and LUMO energy levels the lie within the HOMO-LUMO gap of canonical nucleobases to avoid quenching by PET, but details of the mechanisms for fluorescence turn-on responses of these FBAs remain poorly understood, especially when those responses are specific to base pairing partners. These mechanisms of fluorescence turn-on are important for designing oligonucleotide probes for applications in biochemistry and biophysics and for the design of new nucleoside turn-on probes with complementary properties.

We previously published that ^{DEA}tC derivative is nearly non-emissive as a free nucleoside in aqueous solution but experiences up to a 20-fold fluorescence turn-on effect when base-stacked in double-stranded DNA (dsDNA) and base-paired with guanine. Using solvent isotope effects, we presented evidence that a substantial contributor to ^{DEA}tC's fluorescence turn-on is the ability of the duplex, specifically including Watson-Crick base pairing, to shield ^{DEA}tC from excited-state proton transfer. In the present study, we investigated how ^{DEA}tC's fluorescence turn-on in DNA-RNA duplexes compares with its response in DNA-DNA duplexes and which structural features might explain any observed differences.

Our steady-state fluorescence measurements show that ^{DEA}tC's fluorescence turn-on response in DNA-RNA duplexes is approximately twice as great as in DNA-DNA duplexes, is less sensitive to the identity of neighboring bases, and retains its specificity for base pairing with adenosine. While this large fluorescence turn-on effect, up to approximately 10-fold as compared with the single strand containing ^{DEA}tC or up to 35-fold as compared with the ^{DEA}tC nucleoside, has much potential utility as a bioanalytical tool, in this study we focused on investigating the fluorescence enhancement. We first sought to determine the extent to which ^{DEA}tC perturbs duplex structure. Differences in melting temperature ΔTₘ show that ^{DEA}tC is typically, but not always, moderately destabilizing, particularly in the brightest sequence, GXC. It is less destabilizing than in dsDNA. As we observed previously in dsDNA, CD spectra show that ^{DEA}tC's presence in the DNA-RNA heteroduplex does not change the A-form conformation.

To further gain structural insight into the effects of ^{DEA}tC's presence, we used NMR to determine the conformation of a 10-mer dsDNA duplex GXC′ containing ^{DEA}tC a base pair with G, and compared it with conformation of the canonical duplex GCC′ determined similarly. As anticipated from CD spectra, ^{DEA}tC does not significantly change the B-form conformation of dsDNA, but the duplex is significantly more dynamic at the site of the ^{DEA}tC:G base pair, as evident from the broadness of the NOESY signals. These enhanced dynamics are associated with lower duplex stability, which is corroborated by the lower Tₘ when ^{DEA}tC is present. The NMR structure shows that, on average, ^{DEA}tC base pairs and stacks similarly to cytosine, but with some of its additional arene structure and diethylamino group extending into the major groove, where it is exposed to water and solutes, constrained by the major groove structure.

The classic double-stranded structure of b-form DNA arises from the hydrophobic effect and π-π dispersion energies (E_{π-π}) between stacked nucleobases, with the fidelity of hydrogen bonding being the determinant of matched base pairing. In canonical B-form DNA the observed twist angle (ω) is 36° offset from exact parallel alignment (ω=0°), whereas in A-form DNA the twist between consecutive base-pairs is smaller at ω=33°. Increasing ω in B-form DNA was computationally shown to increase overall duplex thermodynamic stability, principally by reducing Pauli repulsion between neighboring bases. In DNA-RNA A-form hybrids with greater base-pair overlap and reduced physical distance between base pairs, ^{DEA}tC should experience greater π-π interactions that may contribute to an increased fluorescence response. Another likely significant factor is that the polarity in A-form major grooves is predicted to mimic the polarity of 20% H₂O in 1,4-dioxane, about half the estimated polarity of B-form. This less polar environment likely contributes to ^{DEA}tC's enhanced fluorescence.

To better understand how local structure in duplex nucleic acids influences radiative and nonradiative relaxation of ^{DEA}tC's excited state, we performed TCSPC studies on the free nucleoside and the duplexes. In most contexts, ^{DEA}tC exhibits biexponential decay, indicative of two populations. One population has a longer excited state lifetime τ≈7-10 ns, and the second population has a shorter lifetime τ≈2 ns. The population with the longer lifetime is dominant in all cases, except when ^{DEA}tC is “mispaired” with adenine or inosine, in which case the shorter lifetime is dominant. This change to a dominant shorter lifetime explains the lack of fluorescence turn-on response to these base pairings. While we do not have sufficient data to make rigorous structural assignments for these populations, we speculate that the shorter lifetime population may represent (i) a solvated state prone to ESPT quenching that is prevented by Watson-Crick base pairing with three hydrogen bonds, (ii) a tautomeric base pair in the case of ^{DEA}tC:A, or a combination of (i) and (ii) when ^{DEA}tC is base paired with A, or a combination of (i) and (ii).

Last, we performed Stern-Volmer quenching studies to assess how differences in the A-form conformation might leave ^{DEA}tC less exposed to exogeneous quenchers. In addition to a more compressed helix, the A-form duplex as observed in DNA-RNA has deeper major grooves than that of B-form dsDNA. While the major and minor grooves in B-form DNA have isometric depths, the narrower, deeper major grooves of A-form DNA-RNA restrict access and mobility of solvent molecules and some ions. Indeed, quenching rate constants determined from Stern-Volmer analysis and time-resolved fluorescence studies show that quenching by both chloride and iodide is slower in DNA-RNA, although the magnitude of the difference is sequence dependent. This finding supports the premise that reduced access to exogeneous quenchers by virtue of the narrower major groove in the DNA-RNA heteroduplex contributes to ^{DEA}tC's brighter fluorescence in this environment.

Conclusions. In this work, we sought to evaluate the fluorescence turn-on response of DNA strands incorporating ^{DEA}tC upon hybridization to complementary and mismatched RNA. Steady-state fluorescence measurements show that ^{DEA}tC's fluorescence turn-on response to matched RNA oligos is, on average, an 8-fold increase. This response is approximately triple the magnitude of increase upon hybridization with complementary DNA and is dependent on the formation of a “matched” ^{DEA}tC:G base pair. Neighboring bases influence the extent of fluorescence turn-on, but to a lesser degree than is observed when fluorescence turn-on is induced by hybridization to complementary DNA. ^{DEA}tC is, in most sequences, modestly destabilizing, as indicated by depressed melting temperatures, but CD spectroscopy and NMR structure determination show that it does not significantly disrupt native B- and A-form conformations for DNA-DNA and DNA-RNA duplexes, respectively. In the most destabilized sequence context, GXC, NOESY signals indicate that the ^{DEA}tC:G base pair is more dynamic than a canonical C:G base pair. Time-resolved fluorescence studies show that “mispairing” ^{DEA}tC with A or I results in a substantial decrease in fluorescence lifetime <τ> by populating a state that is prone to quenching.

Desolvation of ^{DEA}tC in the base stack and canonical Watson-Crick base pairing is required to minimize this quenching, and results in the fluorescence turn-on. Structural analysis of the A-form in comparison with the B-form and the determination of quenching rate constants by Stern-Volmer analysis and time-resolved fluorescence shows that the compacted A-form duplex, with its narrower major groove structure, provides a less polar environment for ^{DEA}tC, with enhanced base stacking, and reduced access to soluble quenchers. These features together explain ^{DEA}tC's enhanced performance at fluorescence turn-on sensing of matched RNA sequences as compared with matched DNA sequences. Further work is needed to better understand how the electronic and hydrogen bonding interactions between stacked and paired nucleobase analogues gives rise to this and other fluorescent response to local environment in nucleic acids. Given the many advantages of fluorescence turn-on sensing over turn-off sensing, we look forward to many future applications of ^{DEA}tC in biophysical studies and as a fluorescent probe for specific target nucleic acid sequences. The latter application will necessitate the use of longer probe sequences such as 20-mers to enable target specificity in complex biological samples, and it is expected that attention will be needed to avoid probe designs that form stable hairpins or homodimers, which would increase background fluorescence. These studies will be the subject of future reports.

The following Examples is intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Supporting Data for ^{DEA}tC Sensing Probes

TABLE 5
Steady-State fluorescence measurements from ^{DEA}tC in single-stranded
DNA oligonucleotides.

| DNA Sequence | λ_{max,} _{abs} | λ_{max,} ₑₘ | Stokes Shift | | Brightness |
|---|---|---|---|---|---|
| (5′→3′) | (nm) | (nm) | (nm) | Φₑₘ | (M⁻¹ cm⁻¹)^{a} |
| CGCA-GXC-TCG | 425 | 499 | 74 | 0.032 | 86.4 |
| CGCA-GXA-TCG | 425 | 499 | 74 | 0.020 | 54.0 |
| CGCA-TXA-TCG | 417 | 495 | 78 | 0.014 | 37.8 |
| CGCA-CXA-TCG | 420 | 493 | 73 | 0.014 | 37.8 |
| CGCA-CXC-TCG | 421 | 496 | 75 | 0.013 | 35.1 |
| CGCA-AXA-TCG | 415 | 497 | 82 | 0.008 | 21.6 |
| CGCA-GXG-TCG | 415 | 498 | 83 | 0.027 | 72.9 |
| CGCA-CXT-TCG | 413 | 495 | 82 | 0.020 | 54.0 |

The position of ^{DEA}tC is denoted by X. Sequence names are bolded.
^{a}Brightness is calculated using ϵ = 2,700 M⁻¹ cm⁻¹ at 395 nm. Measurements were made in 1 × PBS buffer at pH 7.4.

TABLE 6
Steady-State fluorescence measurements from ^{DEA}tC in DNA-DNA duplexes.

| | | | Stokes | | | |
|---|---|---|---|---|---|---|
| DNA Sequence | λ_{max, abs} | λ_{max, em} | Shift | | | Brightness |
| (5′→3′) | (nm) | (nm) | (nm) | Φₑₘ | ΔTₘª | (M⁻¹ cm⁻¹)^{b} |
| CGCA-GXC-TCG | 348 | 500 | 152 | 0.12 | −14.5 | 324 |
| CGCA-GXA-TCG | 422 | 499 | 77 | 0.050 | −5.1 | 135 |
| CGCA-TXA-TCG | 420 | 495 | 75 | 0.026 | −1.6 | 70.2 |
| CGCA-CXA-TCG | 410 | 494 | 84 | 0.017 | +2.1 | 45.9 |
| CGCA-CXC-TCG | 413 | 496 | 83 | 0.024 | +6.7 | 64.8 |
| CGCA-AXA-TCG | 410 | 498 | 88 | 0.042 | −2.6 | 113.4 |
| CGCA-GXG-TCG | 400 | 499 | 99 | 0.056 | −7.0 | 151.2 |
| CGCA-CXT-TCG | 414 | 492 | 78 | 0.025 | +2.0 | 67.5 |
| AXA abasic mismatch | 421 | 466 | 45 | 0.007 | n.d. | 18.9 |
| AXA adenosine | 417 | 501 | 84 | 0.015 | +9.6 | 40.5 |
| mismatch | | | | | | |

The position of ^{DEA}tC is denoted by X. Sequences names are bolded. Complementary DNA sequences are matched.
ªDuplex stability is calculated by subtracting Tₘ (° C.) of duplexes with natural cytidine from Tₘ (° C.) of duplexes substituting ^{DEA}tC at position X.
^{b}Brightness is calculated using ϵ = 2,700 M⁻¹ cm⁻¹ at 395 nm. Measurements were made in 1 × PBS buffer at pH 7.4.

TABLE 7
Melting temperature of DNA-RNA heteroduplexes containing
^{DEA}tC.

| Sequence | Cytidine Tₘ (+) | ^{DEA}tC Tₘ (±) | ΔTₘ (±) |
|---|---|---|---|
| GXC | 58.36 (0.675) | 51.31 (0.376) | −7.05 (0.77) |
| GXA | 45.20 (1.74) | 42.37 (0.220) | −2.83 (1.8) |
| AXA | 40.53 (0.573) | 41.14 (0.198) | +0.61 (0.60) |
| TXA | 48.27 (0.740) | 44.88 (0.417) | −3.39 (0.85) |
| CXA | 56.72 (0.563) | 49.80 (0.302) | −6.92 (0.64) |
| CXC | 60.30 (0.693) | 59.14 (0.731) | −1.16 (1.0) |
| GXG | 52.03 (0.456) | 46.60 (0.291) | −5.43 (0.54) |
| CXT | 52.26 (0.927) | 53.75 (0.672) | +1.49 (1.1) |

Melting temperatures of duplexes were measured by recording CD signal at 270 nm as a function of temperature. Subtracting Tₘ (C) of duplexes with natural cytidine from Tₘ (° C.) of duplexes containing ^{DEA}tC provided ΔTₘ. Measurements were made in 1 × PBS buffer at pH 7.4.

TABLE 8
Time-resolved fluorescence measurements for DNA-DNA duplexes
containing ^{DEA}tC.

| Sequence | τ₁ (ns) | α₁ | τ₂ (ns) | α₂ | <τ> (ns)^{a} |
|---|---|---|---|---|---|
| TXA | 10.50 | 60% | 1.51 | 40% | 6.93 |
| CXT | 13.12 | 29% | 1.37 | 71% | 4.73 |
| CXA | 9.21 | 19% | 1.91 | 81% | 3.27 |

Time-resolved measurements were performed using TCSPC and λₑₓ = 371 nm. Sequences shown as internal triplet with flanking bases in 5′ to 3′ order.
ªAmplitude average fluorescence lifetime <τ> = Σα₁τ₁.

### Example 2. Synthesis and Methods

Synthesis of oligonucleotide probes. Probe sequences were generated by synthesizing ^{DEA}tC phosphoramidite using published methods (Curr. Protoc. Nucleic Acid Chem. 2018, e59), and incorporated into DNA strands using solid-phase DNA synthesis performed by TriLink Biotechnologies, Inc. (San Diego, CA) or the Keck Oligonucleotide Synthesis facility at Yale University. The HPLC-purified oligonucleotides were characterized by MALDI-TOF mass spectrometry and found to be consistent with calculated masses as shown in Table 9. Complementary RNA and unmodified DNA sequences were purchased from Integrated DNA Technologies, Inc. (San Diego, CA).

TABLE 9
Quantitative analysis of ^{DEA}tC oligonucleotides.

| | Sequence | Calculated | Observed |
|---|---|---|---|
| Sequence Name | (5′...3′) | Mass (g mol⁻¹) | Mass (amu) |
| GXC | CGCA-GXC-TCG | 3166.7 | 3166.5 |
| GXA | CGCA-GXA-TCG | 3190.7 | 3190.1 |
| AXA | CGCA-AXA-TCG | 3174.8 | 3174.2 |
| TXA | CGCA-TXA-TCG | 3165.8 | 3165.2 |
| CXA | CGCA-CXA-TCG | 3150.8 | 3150 |
| CXC | CGCA-CXC-TCG | 3126.8 | 3126 |
| GXG | CGCA-GXG-TCG | 3206.7 | 3206.2 |
| CXT | CGCA-CXT-TCG | 3141.8 | 3140.9 |

Steady-state fluorescence spectroscopy to measure quantum yield of emission. Quantum yield values were measured using the comparative method with quinine sulfate (QS, Φₑₘ=0.54) in 0.1 M H₂SO₄ as a standard. Oligonucleotides were suspended in 1×PBS pH 7.4 prepared with nuclease-free water and measurements were taken in quartz sub-micro cuvettes with a 1.0 cm path length (Starnacell Inc.) at 25° C. Emission spectra were recorded under steady-state conditions using a PTI QuantaMaster QM-400 fluorometer (Horiba Scientific) and absorbance spectra were measured using a Shimadzu UV-1700 Pharmaspec spectrophotometer. ^{DEA}tC oligonucleotides were annealed to 1.2 molar equivalents of RNA complement and heated to 72° C. for 5 min and cooled to room temperature before measuring absorbance and emission. Samples were measured with an initial absorbance near 0.10, approximating 37 μM of fluorophore, at 390 nm. The same wavelength was used as excitation for collecting total visible emission, 405-700 nm. Absorbance and emission spectra recorded with diluted samples for at least six data points. The slopes of integrated emission vs absorbance for QS standard and sample sequences were used in Equation 1 to calculate quantum yield.

$${Φ}_{F} = {Φ}_{r e f} ( \frac{{slope}_{F}}{{slope}_{r e f}} ) {( \frac{{η}_{F}}{{η}_{ref}} )}^{2} \qquad Equation 1$$

Where F denotes values for the fluorophore ^{DEA}tC, quinine sulfate is the reference, and n is the refractive index of 0.10 M H₂SO₄ or 1×PBS. Sequences were measured at least in duplicate and averaged.

Time-dependent fluorescence spectroscopy and excited-state lifetime measurements. Lifetimes were recorded using a Delta Pro™ Fluorescence Lifetime System (Horiba Scientific) with a LED Delta Diode 371 nm excitation laser operating at 25 MHz pulse frequency. Detected photons were collected in the range of 250-650 nm. The instrument response function (IRF) was recorded using dilute colloidal silica (0.30% solution, LUDOX™) and deconvoluted from sample spectra. Time-dependent decays in protonated or deuterated buffer were recorded in a quartz sub-micro cuvette with a 1.0 cm path length (Starnacell Inc.) at 25° C.

Emission plots from time-correlated single photon counting (TCSPC) were fitted to an exponential decay by the maximum entropy method using MemExp version 6.0 with one or two components to calculate the excited-state lifetimes (t). Extremely short lifetime artifacts (where log₁₀ τ<−1.1) were omitted from the model and the fitted emission intensities (I₀) were normalized to 1.00. A two-component model, as shown in Equation 2, was selected if the single-component fitting produced by MemExp was not adequate.

$$I ( t ) = {A}_{1} {e}^{- \frac{t}{{τ}_{1}}} + {A}_{2} {e}^{- \frac{t}{{τ}_{2}}} \qquad Equation 2$$

From the measured quantum yields, the radiative decay rates (kᵣ) and non-radiative rate (kₙᵣ) were calculated using Equation 3 and 4.

$${k}_{f} = \frac{{Φ}_{F}}{τ} \qquad Equation 3$$

$${Φ}_{F} = \frac{{k}_{f}}{{k}_{f} + {k}_{n r}} \qquad Equation 4$$

Duplex structure and thermodynamic stability by circular dichroism spectroscopy. To measure the structure of ^{DEA}tC DNA-RNA heteroduplexes, CD spectra were scanned using 2.5 μM samples at 25° C. in a 0.20 cm quartz cuvette and an Aviv model 420 CD spectrophotometer. CD spectra were averaged from two scans ranging 320 nm to 200 nm, in 1 nm increments. Background 1×PBS spectra were subtracted and raw signal (01) in mdeg was converted to molar elipticity (Δε, M⁻¹ cm⁻¹). Duplex melting temperatures were determined by measuring the absorbance at 270 nm from 20° C. to 80° C. in 1° C. increments. The data were normalized to a two-state model using Equation 5.

$${α}_{F} = \frac{{θ}_{i} - {θ}_{U}}{{θ}_{F} - {θ}_{U}} \qquad Equation 5$$

Where fraction annealed (α_{F}) equals the difference between raw signal minus the signal of fully denatured duplex (θ_{U}), divided by the difference between fully annealed duplex signal (θ_{F}) and denatured duplex signal. The normalized values were fit to a logistic model using OriginLab and Equation 6 to calculate the melting temperature.

$${α}_{F} = \frac{1}{1 + {e}^{- k ( T - {T}_{m} )}} \qquad Equation 6$$

Where T is temperature, Tₘ is the duplex melting temperature, and k is the melting rate.

Fluorescence quenching with halide anions. Stern-Volmer experiments were performed using ^{DEA}tC dsDNA and DNA-RNA samples suspended in 10 mM Na₂HPO₄ pH 7.4 buffer using degassed Ultrapure™ nuclease-free water. Samples were prepared to 37 UM in 150 μL buffer and fluorescence with 395 nm excitation was measured at 25° C. under steady-state conditions. A 4.5 M solution of sodium iodide or sodium chloride (in degassed H₂O) was added in 1 μL increments and the emission was recorded for a total of six data points. The final sample volume amounted to 156 μL corresponding to a final fluorophore concentration of 35.6 μM and a 3.8% molar reduction. The Stern-Volmer quenching efficiency (K_{SV}) was determined from Equation 7 and a linear plot of the data. Fₒ/F is the ratio of emission in the absence of quencher (Q) to the presence of (Q). Calculating the bimolecular quenching rate (k_{q}) is achieved by substituting the unquenched excited-state lifetime in the equation.

$$\frac{{F}_{0}}{F} = 1 + {K}_{S V} [ Q ] = 1 + {k}_{q} τ [ Q ] \qquad Equation 7$$

NMR sample preparation. The oligomers d(CGTAGCCTCC), d(GGAGGCTACG) and d(CGTAGXC TCC) where X=8-diethylamino-tC (8-DEA-tC) were synthesized and purified by Alpha DNA (Canada). Oligomer concentrations (1-2 mM), duplex formation and NMR samples of DNA duplexes 1 (GCC) and 2 (GXC) were identical to those described previously (J. Am. Chem. Soc. 2008, 130 (14), 4869-4878). The following numbering system is used to describe the duplexes in these studies:

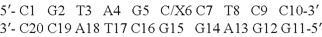

NMR spectroscopy. ¹H NOESY and DQF-COSY spectra were acquired for each duplex in D₂O on a Varian Inova 500 MHz spectrometer using the TPPI method of phase cycling. For structure determination, the GCC duplex signals were best resolved at 27° C., whereas the GXC′ duplex showed significant dynamic behavior near room temperature and signals were best resolved at 15° C. Signal assignments for each duplex were made using NOESY spectra with a mixing time of 300 ms, spectral width of 5913 Hz, 2048 duplex points in t₂ and 512 t₁ increments (zero filled to 2048 on processing). A total of 64 scans were averaged using a recycle delay of 2 s for each t₁ value. Presaturation was applied during the recycling delay and mixing time to suppress residual water signal. Signal assignments were also confirmed using DQF-COSY spectra collected using the same parameters as the NOESY spectra. All spectra for both duplexes were processed with Felix (FelixNMR).

All structural restraints were derived using NOESY spectra in D₂O that were acquired using the TPPI method on a Varian Inova 500 MHz spectrometer. Spectra for quantitative analyses of GCC were collected at 27° C. with mixing times of 200 ms and 50 ms and for GXC at 15° C. with mixing times of 200 ms and 100 ms (2048 duplex points in t₂, 512 t₁ experiments zero filled to 2048 on processing, spectral width of 5913 Hz, and 64 scans for each t₁ value were averaged using a recycle delay of 4 s with presaturation of the HOD resonance). The 2D spectra were apodized with a skewed sine bell function in both dimensions (512 points, phase 60°, skew 0.5 to 0.7 in t₁; 800 points, phase 60°, skew 0.5 to 0.7 in t₂). Prior to Fourier transformation in t₁, the first row of the data matrix was multiplied by a factor of 0.5 to suppress t₁ ridges.

For generating proton distance constraints, the assigned cross peaks of the NOESY spectra were integrated manually using Felix, creating two peak intensity sets for each duplex. The NOEs (uncertainties±20%) were then converted into distances (uncertainties±0.5 Å) classified as very strong (1.8-2.2 Å), strong (2.2-2.8 Å), medium (2.8-4.0 Å), weak (4.0-4.5 Å) or very weak (4.5-5.0 Å) relative to the intensity of the cytosine H5-H6 cross peaks, which are 2.50 Å apart and the sugar C2′H—C2″H cross peaks which are 1.8 Å apart. The lower bounds for all distance restraints were set at 1.8 Å. Dihedral torsion angles (uncertainties±2°) for each duplex were loosely restrained based upon close inspection of the C1′H to C2′H/C2″H region (approximately 5.0-6.4 ppm in F1 and 1.8-2.9 ppm in F2) of the DQF-COSY spectra. The d torsion angle was restrained between 110° and 170° for each nucleotide that had a set of anti-phase multiplet COSY peaks. The overlap of chemical shifts in the GXC duplex hindered detailed analysis of the DQF-COSY spectrum; thus, the d torsion angles for all nucleotides were loosely restrained between 110° and 170° with a lower force constant than used for the GCC duplex. The use of hydrogen bonding restraints (uncertainties±0.2 Å) in our molecular dynamics simulations was justified based on the NMR spectra of the imino protons acquired at 20° C. for both duplexes in H₂O. All DNA bases in both duplexes had observable imino N—H proton peaks and displayed broadening behavior that is characteristic of Watson-Crick hydrogen bonded base pairing as temperature increased. A total of 365 constraints were applied to GCC (including Watson-Crick hydrogen bonding constraints, 221 NMR-derived distance and torsion restraints). For GXC, a total of 302 constraints were applied (including Watson-Crick hydrogen bonding constraints, 163 NMR-derived distance and torsion restraints).

Molecular dynamics calculations. The solution structures of both duplexes were calculated using methods previously described. All forcefield parameters for 8-DEA-tC were calculated using Gaussian. The SANDER module of AMBER 19 was used to perform all computational analyses, including energy minimization and restrained molecular dynamics (rMD) calculations. The NAB molecular manipulation language was used to create a series of 40 starting structures for each duplex differing in the four helical parameters inclination, rise, twist, and x-displacement. The starting duplex structures were then produced following 1000 steps of restrained steepest descent energy minimization using hydrogen bonding restraints with a force constant of 100 kcal mol⁻¹ Å⁻² and intermolecular NMR restraints with a force constant of 32 kcal mol⁻¹ Å⁻², followed by slow equilibration to 0 K. Both sets of structures were then subjected to two rounds of restrained simulated annealing. In the first round, the temperature was increased gradually from 0 K to 600 K over 5 ps and lowered back to 0 K over the next 15 ps, while all 365 constraints (GCC) or 302 constraints (GXC) were increased over 3 ps to full strength, where they remained for an additional 17 ps of rMD. The refinements were completed with a final cycle of rMD with the same conditions as the first. In each 20 ps round of simulated annealing, the force constant for all NMR-derived distance constraints was increased linearly from 0 to 32 kcal mol⁻¹ Å² over 3 ps, remaining at 32 kcal mol⁻¹ Å⁻² for the final 17 ps. The force constants for Watson-Crick hydrogen bonding were held constant at 32.0 kcal mol⁻¹ Å⁻² for both rounds. The force constants for the torsion restraints were held constant at 20 kcal mol⁻¹ Å⁻² for both rounds. Helical parameters for the final structures for both duplexes were then determined using CURVES5+.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A nucleic acid comprising formula I: 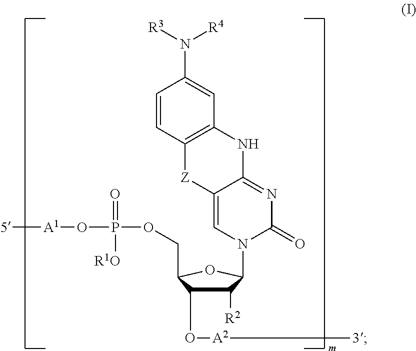
wherein
A¹ is a nucleotide comprising guanine (G), adenine (A), cytosine (C), thymine (T), or a combination thereof;
A² is a nucleotide comprising cytosine (C), adenine (A), guanine (G), thymine (T) or a combination thereof;
R¹ is H or —(C₁-C₆)alkyl;
R² is H or OH;
R³ and R⁴ are each independently —(C₁-C₆)alkyl or H; or
R³ and R⁴ taken together with the nitrogen atom to which they are attached form a 3- to 8-membered heterocycloalkyl, wherein the heterocycloalkyl is unsubstituted or substituted;
Z is S or O;
5′ and 3′ are each independently oligo- or polynucleotides; and
m is 1 to about 100.

2. The nucleic acid of claim 1, wherein R³ and R⁴ are both methyl, ethyl, or propyl.

3. The nucleic acid of claim 1, wherein R³ and R⁴ taken together with the nitrogen atom to which they are attached form:

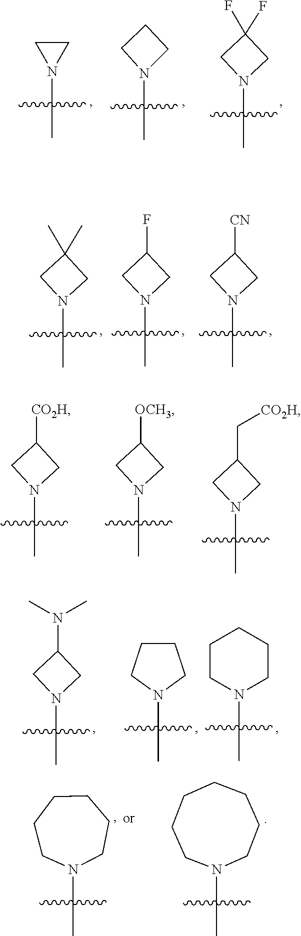

4. The nucleic acid of claim 1, wherein formula I is represented by formula IA: 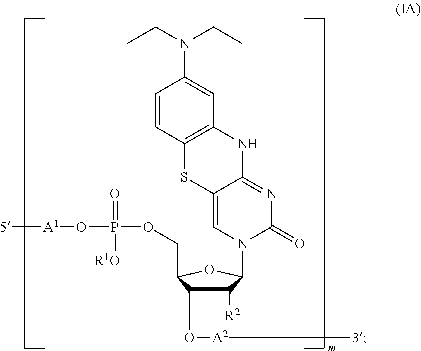
wherein
A¹ is a nucleotide comprising adenine (A), cytosine (C), guanine (G), thymine (T), or a combination thereof;
A² is a nucleotide comprising adenine (A), cytosine (C), guanine (G), thymine (T) or a combination thereof;
R¹ is H or —(C₁-C₆)alkyl;
R² is H or OH;
5′ and 3′ are each independently oligo- or polynucleotides; and
m is 1 to about 100.

5. The nucleic acid of claim 1, wherein R¹ and R² are H.

6. The nucleic acid of claim 1, wherein formula I is represented by formula IB: 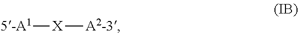
wherein X is the moiety between A¹ and A² of formula I: 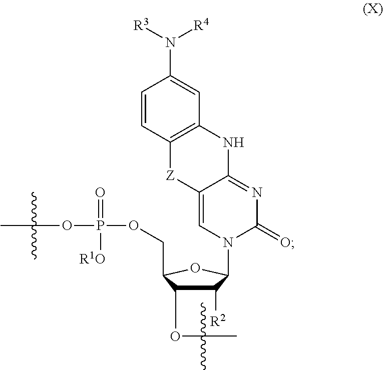
and
the nucleic acid comprises: GXC, GXA, TXA, CXA, CXC, AXA, GXG, or CXT.

7. The nucleic acid of claim 3, wherein the nucleic acid comprises:

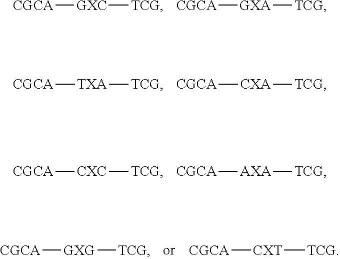

8. The nucleic acid of claim 1, represented by formula IB: 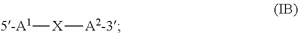
wherein,
X is the moiety between A¹ and A² of formula I; and
the nucleic acid comprises: TXG, GXT, CXG, AXG, AXT, AXC, TXC, or TXT.

9. The nucleic acid of claim 1, wherein A¹ comprises G and A² comprises A, C, G, T, or a combination thereof; or
A¹ comprises C and A² comprises A, C, G, T, or a combination thereof.

10. The nucleic acid of claim 1, wherein A¹ comprises T and A² comprises A, C, G, T, or a combination thereof; or
A¹ comprises A and A² comprises A, C, G, T, or a combination thereof.

11. The nucleic acid of claim 1, wherein A² comprises G and A¹ comprises A, C, G, T, or a combination thereof; or
A² comprises C and A¹ comprises A, C, G, T, or a combination thereof.

12. The nucleic acid of claim 1, wherein A² comprises T and A¹ comprises A, C, G, T, or a combination thereof; or
A² comprises A and A¹ comprises A, C, G, T, or a combination thereof.

13. The nucleic acid of claim 1, wherein m is about 5 to about 25.

14. A method for differentiating nucleic acid sequences, comprising:
a) hybridizing the nucleic acid of claim 1 to a complementary DNA or RNA sequence to form a homoduplex- or heteroduplex-nucleic acid;
b) detecting a fluorescent signal; and
c) quantifying the fluorescent signal, wherein the formed homoduplex- or heteroduplex-nucleic acid increases the fluorescent signal in comparison to a single stranded form of formula I;
wherein the DNA or RNA sequence can thereby be differentiated from a non-complementary sequence of DNA or RNA.

15. The method of claim 14, wherein formula I is represented by formula IB: 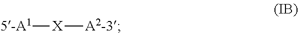
wherein X is the moiety between A¹ and A² of formula I: 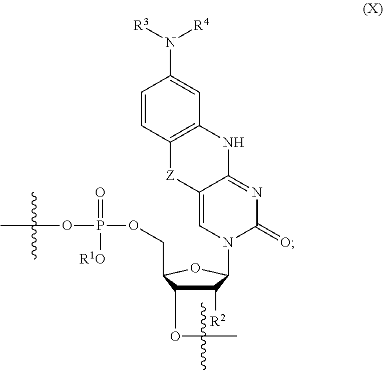
and
the nucleic acid comprises: GXC, GXA, TXA, CXA, CXC, AXA, GXG, or CXT.

16. The method of claim 14, wherein the tricyclic cytidine moiety of formula I is based-paired with guanosine or a guanine base of the complementary DNA or RNA sequence.

17. The method of claim 14, wherein a heteroduplex-nucleic acid is formed with a complementary RNA sequence.

18. The method of claim 14, wherein the quantified fluorescence has an increase in emission quantum yield (Φₑₘ) of about 5-fold or higher.


SEQUENCE LISTING (10 sequences)

SEQ ID NO: 1 - DNA, 10 bp, synthetic construct
  source 1..10: mol_type = other DNA
  modified_base 6: mod_base = OTHER; note = c is 8-Diethylamino-tricyclic-2'-deoxy-beta-D-ribonucleoside
cgcagcctcg

SEQ ID NO: 2 - DNA, 10 bp, synthetic construct
  source 1..10: mol_type = other DNA
  modified_base 6: mod_base = OTHER; note = c is 8-Diethylamino-tricyclic-2'-deoxy-beta-D-ribonucleoside
cgcagcatcg

SEQ ID NO: 3 - DNA, 10 bp, synthetic construct
  source 1..10: mol_type = other DNA
  modified_base 6: mod_base = OTHER; note = c is 8-Diethylamino-tricyclic-2'-deoxy-beta-D-ribonucleoside
cgcatcatcg

SEQ ID NO: 4 - DNA, 10 bp, synthetic construct
  source 1..10: mol_type = other DNA
  modified_base 6: mod_base = OTHER; note = c is 8-Diethylamino-tricyclic-2'-deoxy-beta-D-ribonucleoside
cgcaccatcg

SEQ ID NO: 5 - DNA, 10 bp, synthetic construct
  source 1..10: mol_type = other DNA
  modified_base 6: mod_base = OTHER; note = c is 8-Diethylamino-tricyclic-2'-deoxy-beta-D-ribonucleoside
cgcaccctcg

SEQ ID NO: 6 - DNA, 10 bp, synthetic construct
  source 1..10: mol_type = other DNA
  modified_base 6: mod_base = OTHER; note = c is 8-Diethylamino-tricyclic-2'-deoxy-beta-D-ribonucleoside
cgcaacatcg

SEQ ID NO: 7 - DNA, 10 bp, synthetic construct
  source 1..10: mol_type = other DNA
  modified_base 6: mod_base = OTHER; note = c is 8-Diethylamino-tricyclic-2'-deoxy-beta-D-ribonucleoside
cgcagcgtcg

SEQ ID NO: 8 - DNA, 10 bp, synthetic construct
  source 1..10: mol_type = other DNA
  modified_base 6: mod_base = OTHER; note = c is 8-Diethylamino-tricyclic-2'-deoxy-beta-D-ribonucleoside
cgcaccttcg

SEQ ID NO: 9 - DNA, 10 bp, synthetic construct
  source 1..10: mol_type = other DNA
cgtagcctcc

SEQ ID NO: 10 - DNA, 10 bp, synthetic construct
  source 1..10: mol_type = other DNA
ggaggctacg